(19) United States
(12) Patent Application Publication
(10) Pub. No.: US 2025/0107938 A1
(43) Pub. Date: Apr. 3, 2025

(54) WOUND TREATMENT DEVICES, KITS, SYSTEMS, AND METHODS

(71) Applicant: KERECIS LIMITED, Isafjordur (IS)

(72) Inventors: Gudmundur Fertram SIGURJONSSON, Isafjordur (IS); Dan MOORADIAN, Isafjordur (IS); Tryggvi Orn GUNNARSSON, Isafjordur (IS); Sigurdur HANNESSON, Isafjordur (IS)

(21) Appl. No.: 18/901,643

(22) Filed: Sep. 30, 2024

Related U.S. Application Data
(60) Provisional application No. 63/586,931, filed on Sep. 29, 2023.
(60) Provisional application No. 63/541,661, filed on Sep. 29, 2023.

(51) Int. Cl. A61F 13/02

(52) U.S. Cl. CPC A61F 13/0233; A61F 13/0243

(57) ABSTRACT

A wound treatment device is provided that includes a skin substitute or attached with an absorbent core that conforms to the shape of the wound bed. The wound treatment device is adapted to be applied to a wound such that the skin substitute is in contact with the wound bed and the absorbent core conforms to and fills the space of the wound bed.

### CROSS-REFERENCE TO RELATED APPLICATIONS

This application claims the benefit of and priority to U.S. Provisional Patent Application 63/586,931, entitled “Wound Treatment”, filed at the United States Patent Office on Sep. 29, 2023, which is incorporated herein by reference and U.S. Provisional Patent Application 63/541,661, entitled “Wound Dressing”, filed at the United States Patent Office on Sep. 29, 2023, which is also incorporated herein by reference.

### FIELD OF THE DISCLOSURE

The disclosure relates generally to wound treatment devices for stabilizing, protecting, and/or healing damaged tissue and methods for making the same.

### BACKGROUND

The skin is the largest organ in the human body and acts a protective barrier against physical injury (e.g., wounds) and foreign bodies. A wound is defined as a breakage in the continuity of the skin. When the skin is damaged, the wound can become contaminated and infected by microorganisms. Wounds can be classified into two types: acute and chronic. Acute wounds heal normally in an efficient manner and follow the natural phases of hemostasis, inflammation, proliferation, and remodeling. Chronic wounds do not follow these natural phases and instead remain inflamed, often getting “stalled” in one of the natural phases of wound healing. If left untreated, chronic wounds can develop and even lead to fatal complications.

It is desirable for wound treatment devices to utilize the natural principles of wound healing. Namely, it is desirable for the wound treatment device to provide a moist environment to optimize healing. However, a moist, warm environment created by wounds, e.g., ulcers, also provides an ideal environment for bacterial growth. A large amount of drainage can indicate infection, whereas a reduction in the amount of drainage can indicate inadequate arterial circulation or that an infection is resolving. Thus, there is a need for proper drainage of wound exudate to promote proper healing.

Foam dressings (e.g., Lyofoam™, Allevyn™, Tielle™) provide adequate absorbency and thermal insulation, but their strong adhesive properties can cause dermatitis. A frequent problem when treating exuding wounds is maceration. Usually, the absorbent part of the dressing is optimized to substantially vertical absorption, so that the skin surrounding the wound is not exposed to the exudate in order to avoid maceration of this healthy, but fragile skin. However, these properties are limiting the absorption capacity of the dressing to the part of absorbent material being directly over the wound. Barrier cream/skin conditioning paste, such as zinc paste, may be used on the surrounding skin in order to avoid the maceration, but the paste will often inhibit both the adhesive tack of the dressing as well as the ability of absorbing exudate. Additionally, gaps between the foam layer and wound bed lead to pools of exudate that promote bacterial growth and increase the risk of infection.

Biatain® Silicone Ag is a commercially available wound dressing that fills the gap between the wound bed and dressing and reduces exudate pooling to promote optimal healing conditions. Biatain® Silicone Ag has been shown to kill mature biofilms (P. aeruginosa) and to prevent biofilm formation. Due to its strong antimicrobial activity, silver (Ag) is a commonly used adjunct in wound care. However, it also has the potential to impair healing by exerting toxic effects on keratinocytes and fibroblasts. In addition to disputable impacts, e.g., limited by hypersensitivity, antimicrobial silver nanoparticles in dressings require increased manufacturing costs.

Regarding another principle of wound healing, it is desirable for the dressing or treatment to aid in the management of the infection itself. The field of regenerative medicine focuses on the growth and replacement of damaged tissue by facilitating the growth and proliferation of host cells at the site of injury, thereby resulting in faster healing time and a more permanent solution. A variety of human, animal, and synthetic materials are currently described or used in medical procedures to augment, repair, or correct tissue defects.

U.S. Pat. No. 8,613,957, granted Dec. 24, 2013, and incorporated herein by reference in its entirety, describes an exemplary scaffold material for wound care and/or other tissue healing applications that is constituted of a decellularized extracellular matrix from fish skin. The decellularized fish skin product provides an intact scaffold for support for ingrowth of endothelial and/or epithelial cells. The decellularized fish skin scaffold material is also biocompatible and thus can be integrated by the host. MariGen™ is a commercially available skin substitute made from the minimally processed skin of wild-caught Atlantic cod originating from Iceland. The fish skin is structurally alike to human skin with three basic layers including epidermis, dermis, and hypodermis and contains proteins, lipids, fatty acids, and other bioactive compounds that are homologous to human skin. MariGen™ is used for the management of chronic wounds such as diabetic wounds, pressure ulcers, vascular ulcers, and draining wounds commonly treated in the private office and wound care centers.

The inventors of the present disclosure discovered that integrating a biological scaffold material with an absorbent core that conforms to the shape of the wound bed provides an improved wound treatment device that synergistically maintains moisture in the wound bed and promotes healing and tissue regeneration.

### SUMMARY

The present disclosure is directed to an improved, integrated wound treatment device for supporting tissue regeneration of wounds. The wound treatment device comprises a biological skin substitute integrated with an absorbent core that conforms to the shape of the wound bed.

The skin substitute is integrated or attached to the absorbent core. Integration of the skin substitute with the absorbent core may include chemical bonding or discrete, segmentized skin substitute portions being formed within the absorbent core. Attachment of the skin substitute to the absorbent core may include glues, adhesives, stitching, weaving, or introducing one or more layers with cohesive properties. The wound treatment device is adapted to be applied to a wound such that the skin substitute is in contact with the wound bed and the absorbent core fills the space of the wound bed.

The absorbent core comprises a shape-conformable polymer foam layer that adapts to the shape of the wound bed. The foam may comprise polyurethane or another conformable foam polymer that absorbs wound exudate, conforms to the shape of the wound bed, and reduces exudate pooling. In an embodiment, the absorbent core comprises microcapillaries to facilitate vertical absorption of exudate. The term vertical absorption is defined as absorption of fluid in the direction substantially perpendicular to the plane defined by the adjacent wound skin. The microcapillaries are preferably formed as channels comprising lateral pockets for containing fluid uptake or retaining exudate. It will be understood that in the context of the invention, the terms fluid, moisture, and exudate are used interchangeably regarding wounds and wound dressings. In an embodiment, the microcapillaries are formed within the exudate locking or receptacle layer. In an alternative embodiment, the microcapillaries are formed in both a conformable foam layer and receptacle layer.

A backing layer may be present in all of the embodiments of the dressing of the present invention. Preferably the backing layer is conformable to animal (inclusive of human) anatomical surfaces, is impermeable to liquid, and is vapor permeable. The backing layer, in combination with the absorbent core, may be constructed to define a reservoir or receptable layer therebetween when the dressing is in an expanded moisture-laden state. The receptacle layer is beneficial for locking away absorbed exudate and preventing skin maceration. In embodiments where the backing layer does not permit the passage of a liquid or exudate, moisture in the absorbed exudate passes through the backing layer in a vapor form into the atmosphere. In certain embodiments, the backing layer comprises an air-tight material for use in combination with negative pressure treatment of the wound.

The wound treatment device comprises a release liner for handling the wound treatment device in an aseptic manner. The release liner protects the skin and wound contacting surfaces of the skin substitute, and any surrounding adhering layer, from contamination before application of the wound treatment device. In an embodiment, the release liner is transparent to allow for observation of the skin substitute. The release liner preferably comprises at least one flap to allow for convenient separation of the release liner from both the skin substitute and silicone layer. Providing the release liner with flaps or a slit allows for the release liner to be easily grasped and pealed from the wound treatment device before application to the wound. In an embodiment, the release liner comprises a first layer and a second layer, wherein the second layer overlaps a portion of the first layer of the release liner. The portion of the first layer is thus disposed between the second layer and the acellular dermal matrix.

In another preferred embodiment, a wound treatment device is provided comprising: a skin substitute configured to be placed in contact with a wound; a contact film arranged in contact with the skin substitute between the absorbent core and the skin substitute; and a replaceable portion arranged overlaying the contact film, the replaceable portion including an absorbent core configured to absorb exudate from the wound and a top film overlying the absorbent core, such that the absorbent core is arranged between the contact film and the top film, wherein the replaceable portion is separable from the contact film such that the replaceable portion may be removed from the contact film without disrupting or removing the skin substitute.

In an embodiment, the skin substitute is in direct contact with a polymer contact layer comprising a mildly cohesive silicone. The wound treatment device is configured to be applied to a wound such that the skin substitute is in contact with the wound and the polymer contact layer is layered between the skin substitute and absorbent core. The polymer contact layer is integrally bonded to the absorbent core and removably adhered to the skin substitute. In embodiments where the polymer contact layer comprises or consists of a silicone layer, the silicone layer is advantageously in direct contact with the skin graft material and is biocompatible with the skin. Additionally, the polymer contact layer prevents exudate from escaping, or transporting back into the wound bed, under compression. In an embodiment, the silicone layer provides a gentle adhering bond the skin substitute for proper fixation and painless separation after healing has occurred. The silicone layer has sufficient tackiness to negate a need for an adhesive between the silicone layer and the skin graft material. An exemplary polymer contact layer is described in greater detail in U.S. Provisional Application No. 63/541,661, filed on Sep. 29, 2023 entitled “Wound Dressing”, which is incorporated herein by reference in its entirety.

After application of the skin graft material to a wound, and after a period of time has elapsed, the polymer contact layer is adapted to conveniently separate from the skin substitute. In an embodiment, the period of time may be for up to fourteen days or may be for a time window that allows for cellular ingrowth and tissue regeneration at the wound site. The polymer contact layer preferably has a predetermined porosity to permit wound exudate to pass through the thickness of the polymer contact layer toward the absorbent core. The predetermined porosity defines a distribution of apertures over a surface area of the polymer contact layer. The distribution of apertures is configured to provide sufficient transfer of wound exudate to the absorbent core while allowing sufficient contact between the surface area of the silicone layer and the skin substitute and/or peri-wound skin to maintain a gentle adherence.

As noted above, the wound treatment device may comprise discrete, segmentized skin substitute portions that are formed within the absorbent core. By punching segments of the skin substitute partly into the absorbent core, discrete portions of the skin substitute are distributed about the wound contacting surface of the wound treatment device. These discrete portions of the fish skin are applied in a dried form, meaning that rehydration of the skin substitute is not needed prior to use. In an embodiment, the discrete portions are formed as particles for individual integration with the host at the wound site. The discrete portions may be in direct contact with the absorbent core or on the wound contacting surface of the polymer contact layer.

In an embodiment, the skin substitute includes bovine, porcine, biosynthetic, and fish skin. Although studies have provided evidence of fish skin grafts being a preferred embodiment, a bovine, porcine, or biosynthetic skin could of course still be used as an effective skin substitute according to the present disclosure, and under some conditions or considerations, may also be a preferred embodiment of a skin substitute as contemplated in the current disclosure. In an embodiment, the skin substitute, or skin graft material, is an acellular dermal matrix. The acellular dermal matrix is a biological scaffold-type material, such as a fish skin product, which is configured to be absorbed and grown into by the skin cells as the wound heals. The acellular dermal matrix may comprise decellularized, lyophilized fish skin. In an embodiment, the acellular dermal matrix comprises lipids from a lipid layer of the decellularized fish skin to enhance the natural healing process of human skin. In an embodiment, the skin substitute comprises an extracellular matrix product in a three-dimensional form of particles, or a sheet, or a mesh to provide enhanced structural support and allow for improved tissue regeneration and cellular ingrowth. In a scaffold form, the acellular dermal matrix is preferably rehydrated before application; however, the scaffold may also be used in dried form. Additionally, the skin substitute may be fenestrated. The fenestration may define at least one slot that extends through the thickness of the skin substitute. Providing the skin substitute with fenestrations allows for proper wound drainage of exudate during healing. The fenestrations also provide greater flexibility in conforming to the wound bed surface.

Numerous other advantages and features of the present invention will become more readily apparent from the following detailed description of the invention, the accompanying examples, drawings and the appended claims.

### BRIEF DESCRIPTION OF THE DRAWINGS

Features, aspects, and advantages of the presently disclosed technology may be better understood concerning the following description, appended claims, and accompanying drawings. The features shown in the drawings are for purposes of illustration, and variations, including different or additional features and arrangements thereof, are possible.

FIG. 1 illustrates an exploded view of an embodiment of a wound treatment device;

FIG. 2 illustrates a cross sectional view of an embodiment of a wound treatment device;

FIG. 3 illustrates a cross sectional view of an embodiment of a wound treatment device applied to a wound;

FIG. 4 illustrates a perspective view of an exemplary skin substitute for a wound treatment device;

FIG. 5A illustrates a perspective view of an exemplary polymer contact layer for a wound treatment device;

FIG. 5B illustrates a perspective view of another exemplary polymer contact layer for a wound treatment device;

FIG. 6 illustrates a perspective view of an exemplary conformable foam layer for a wound treatment device;

FIG. 7 illustrates a perspective view of an exemplary receptacle layer for a wound treatment device;

FIG. 8 illustrates an exemplary microcapillary of an absorbent core for a wound treatment device;

FIG. 9 illustrates a perspective view of an embodiment of the wound treatment device according to the present disclosure;

FIG. 10 illustrates a cross sectional view of an embodiment of the wound treatment device according to the present disclosure;

FIG. 11 illustrates a negative pressure wound treatment device application of the wound treatment device of the present disclosure;

FIG. 12A illustrates a cross sectional view of an embodiment of a wound treatment device;

FIG. 12B illustrates a cross sectional view of another embodiment of a wound treatment device;

FIG. 13A illustrates a schematic exploded cross sectional view of an embodiment of a wound treatment device;

FIG. 13B illustrates a schematic exploded perspective exploded view of an embodiment of a wound treatment device;

FIG. 14A illustrates a schematic cross sectional view of a contact film of an embodiment of a wound treatment device;

FIG. 14B illustrates a schematic cross sectional view of a top film of an embodiment of a wound treatment device;

FIGS. 15A, 15B, 15C, 15D, 15E, and 15F show a progression of steps of a use of the wound treatment device according to a preferred embodiment;

FIG. 16 illustrates a schematic cross sectional view of an embodiment of a wound treatment device applied to a wound;

FIGS. 17A, 17B, 17C, 17D, 17E, and 17F show plan views of embodiments of wound treatment device of different size and shape;

FIGS. 18A, 18B, 18C, 18D, 18E, 18F, and 18G show a progression of steps of a use of the wound treatment device according to a preferred embodiment

FIG. 19A illustrates a schematic exploded cross sectional view of an embodiment of a wound treatment device;

FIG. 19B illustrates a schematic exploded perspective exploded view of an embodiment of a wound treatment device;

FIG. 19C illustrates a schematic cross sectional view of a contact film of an embodiment of a wound treatment device;

FIG. 19D illustrates a schematic cross sectional view of a contact film of an embodiment of a wound treatment device applied to a wound bed;

FIG. 20A illustrates a schematic cross sectional view of a contact film of an embodiment of a wound treatment device;

FIG. 20B illustrates a schematic cross sectional view of a contact film of an embodiment of a wound treatment device applied to a wound bed;

FIG. 21 illustrates a schematic cross sectional view of a contact film of an embodiment of a wound treatment device; and

FIG. 22 illustrates a schematic cross sectional view of a contact film of an embodiment of a wound treatment device.

The drawings are to illustrate exemplary implementations and are not drawn to scale. It is understood that the inventions are not limited to the arrangements and instrumentalities shown in the drawings.

### Definitions

Descriptions of the following terms are provided for further ease of understanding the embodiments of the disclosed wound treatment device. As used herein, the term “wound treatment device” means a multi-layered, integrated wound dressing. The term “wound dressing” means an interactive product (e.g., bandage, covering, compress, protective layer) which can be utilized in management of a wound.

The term “skin substitute” means a group of elements or materials that enables the temporary or permanent occlusion of a wound.

The terms “acellular,” “decellularized,” “decellularized fish skin,” and the like as used herein refer to a fish skin from which a substantial amount of cellular and nucleic acid content has been removed leaving a complex three-dimensional interstitial structure of ECM. In embodiments, “decellularized fish skin” may further entail fish skin which, in addition to the complex three-dimensional interstitial structure of ECM absent a substantial amount of cellular and nucleic acid content, includes omega 3 polyunsaturated fatty acids (PUFAs).

The terms “extracellular matrix” or “ECM” as used herein refer to the non-cellular tissue material present within the fish skin that provides structural support to the skin cells in addition to performing various other important functions. The ECM described herein does not necessarily include matrix material that has been constituted or re-formed entirely from extracted, purified, or separated ECM components (e.g., collagen). But in some embodiments, an ECM used as a skin substitute may include matrix material that has been constituted or re-formed entirely from extracted, purified, or separated ECM components (e.g., collagen).

The term “treatment” is intended to be understood by its common dictionary definition. That is, the term “treatment” broadly includes medical care devices (i.e., bandages and dressings) and/or medicaments given to a patient for an illness or injury. As should be appreciated by those having skill in the art, a “treatment” includes the use of a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

The term “wound” as used herein is intended to encompass tissue injuries generally. Thus, the term “wound” includes those injuries that cause, for example, cutting, tearing, and/or breaking of the skin such as lacerations, abrasions, incisions, punctures, avulsions, or other such injuries. Wounds may be described by any size, shape, or magnitude of the wound. For example, a paper cut is exemplary of a small, straight incision of relatively little magnitude, whereas a concussive blast resulting in a major laceration covering one or multiple body parts is exemplary of a relatively larger wound of greater magnitude. Each of the foregoing examples, however, fall within the scope of the term “wound,” as used herein.

The term “wound” additionally includes damage to underlying tissue, such as that caused by traumatic injury. As such, the term “wound” is intended to include a combination of multiple different wounds. For example, a traumatic amputation caused by an explosive blast may generally be referred to as a wound even though it is a compilation of a host of different lacerations, abrasions, avulsions, and punctures. Additionally, any underlying tissue damage resulting from the aforementioned explosive blast may further be encompassed within the understanding of this reference to a wound. The term “wound” is also intended to encompass tissue injuries caused by burns (e.g., thermal and/or chemical burns). Further, the term “wound” is also intended to encompass injuries resulting from, for example, diabetic foot ulcers, venous leg ulcers, surgical operations, pressure ulcers, and other causes.

The term “biocompatible” refers to a material that is substantially non-toxic in the in vivo environment of its intended use, and that is not substantially rejected by the patient's physiological system (i.e., is non-antigenic).

The term “polymer contact layer” is a polymeric covering, that may be a single-layer structure or a multilayer structure, that is a generally impermeable to fluids and preferably impermeable to fluids and bacteria. The polymer contact layer may allow moisture permeation. The polymer contact layer is provided with apertures or is sufficiently porous to allow passage of wound exudate through the polymer contact layer.

The term “silicone layer” means a soft, medical-grade silicone gel-like layer designed to act as a gentle adhesive contact layer to the skin surrounding the wound.

The term “fenestration” means perforations, slits, slots, apertures, or openings that extend through the thickness of a layer.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

### DETAILED DESCRIPTION

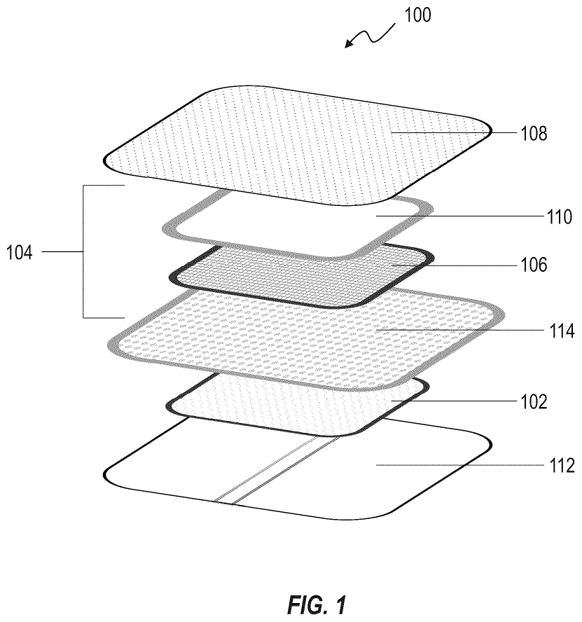

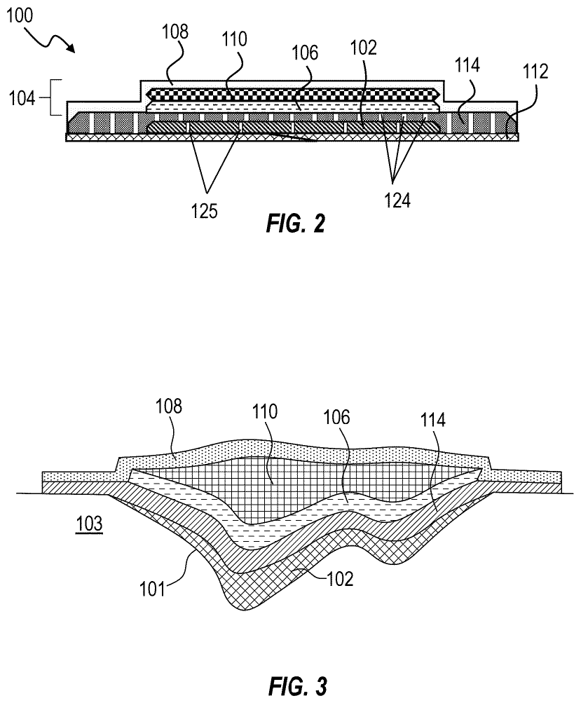

FIGS. 1 and 2 each depict a schematic of wound treatment device 100 according to a first embodiment. FIG. 1 illustrates an exploded view of an embodiment of a wound treatment device, and FIG. 2 FIG. 2 illustrates a cross sectional view of an embodiment of a wound treatment device. In the embodiment of FIGS. 1 and 2, the wound treatment device 100 is a wound dressing comprising a skin substitute 102 used to support tissue regeneration of wounds. The wound treatment device 100, as the other wound treatment devices described and shown herein, is suitable for management of the following wounds: partial and full thickness wounds, pressure ulcers, venous ulcers, chronic vascular ulcers, diabetic ulcers, trauma wounds (e.g., abrasions, lacerations, partial thickness burns, skin tears), and surgical wounds (e.g., donor sites/grafts, post-Mohs surgery, post-laser surgery, podiatric, wound dehiscence).

The wound treatment device 100 comprises a skin substitute 102 coupled to an absorbent core 104. The skin substitute 102 is adhered to a polymer contact layer 114 that is disposed between the skin substitute 102 and absorbent core 104. The absorbent core 104 may comprises a conformable foam layer 106 for conforming to a wound bed of the wound to be treated and a receptacle layer 110 for absorbing and containing wound exudate. The wound treatment device 100 further may include a backing layer 108 to protect the wound bed 101 and peri-wound skin 103 and to also protect the lower layers of the wound treatment device 100 from damage and contamination. The wound treatment device 100 also comprises a release liner 112 for aseptic handling of the wound treatment device 100, which is configured to be removed before application at the wound site.

FIG. 3 illustrates a cross sectional view of the wound treatment device 100 applied to the wound bed 101 and peri-wound skin 103. The skin substitute 102 is arranged to be placed into direct contact with the wound bed 101 when the wound treatment device 100 is applied to the wound such that the skin substitute conforms to the terrain of the wound, to facilitate tissue regeneration and healing. In an embodiment, the polymer contact layer 114 above the skin substitute 102 comprises a gently adhering silicone layer that conforms to the shape of the wound bed 101. The polymer contact layer 114 facilitates the transportation of wound exudate via apertures (discussed below) and protects the skin substitute 102 and peri-wound skin 103. In an embodiment, the polymer contact layer 114 prevents absorbed wound exudate from transporting back to the wound bed 101. The conformable foam layer 106 works to fill the gap between the wound treatment device 100 and wound bed 101 to reduce exudate pooling and transporting the exudate to the receptacle layer. As observed in FIG. 3, the receptacle layer 110 expands to entrap or lock in the absorbed exudate below the backing layer 108. The unique layers, and relationships between layers, of the wound treatment device 100 are described in greater detail below.

Skin substitutes 102 may be considered broadly as a group of elements or materials that enable the temporary or permanent occlusion of a wound, and preferably that promote skin growth. Skin substitutes 102 can generally be divided into biological skin substitutes, synthetic skin substitutes, or a hybrid skin substitute that includes biological and synthetic skin substitutes. Biological skin substitutes often have a more intact extracellular matrix structure, while the synthetic skin substitutes can be synthesized on demand and can be modulated for specific purposes. Biological skin substitutes and synthetic skin substitutes each have advantages and disadvantages. The biological skin substitutes may allow the construction of a more natural new dermis and allow excellent re-epithelialization characteristics due to the presence of a basement membrane. Synthetic skin substitutes may be chemically synthesized and provide the advantages of increased control over scaffold composition. Synthetic skin substitutes include synthetic biolayers including, for example, a synthesized collagen or protein-based matrix or a collagen or protein-based components combined with silicone components. Hybrid skin substitutes may be partly synthesized or produced by living cells and partly chemically synthesized.

Whether biological, synthetic, or hybrid skin substitutes are used, the object of using skin substitutes is to provide an effective, timely, and scar-free wound healing with as much return to the functions of the skin before the wound event. Examples of such skin substitutes are described in U.S. Patent Publication No. 2022/0313873, based on U.S. patent application Ser. No. 17/703,650, filed Mar. 24, 2022, which is incorporated herein by reference in its entirety.

Often, in the cases of significant wounds, such as wounds extending over large areas or in deep wounds, or large or severe burn wounds, or in the case of chronic wounds, skin substitutes are often used to aid in the healing process of the wound and to more quickly restore at least some of the above-noted functions of healthy skin. Skin substitutes may be considered broadly as a group of elements or materials that enable the temporary or permanent occlusion of a wound. Skin substitutes can generally be divided into biological skin substitutes, synthetic skin substitutes, or a hybrid skin substitute that includes biological and synthetic skin substitutes.

Biological skin substitutes often have a more intact extracellular matrix structure, while the synthetic skin substitutes can be synthesized on demand and can be modulated for specific purposes. Biological skin substitutes and synthetic skin substitutes each have advantages and disadvantages. The biological skin substitutes may allow the construction of a more natural new dermis and allow excellent re-epithelialization characteristics due to the presence of a basement membrane. Synthetic skin substitutes may be chemically synthesized and provide the advantages of increase control over scaffold composition. Synthetic skin substitutes include synthetic biolayers including, for example, a synthesized collagen or protein-based matrix or a collagen or protein-based components combined with silicone components. Hybrid skin substitutes may be partly synthesized or produced by living cells and partly chemically synthesized.

Whether biological, synthetic, or hybrid skin substitutes are used, the object of using skin substitutes is to provide an effective, timely, and scar-free wound healing with as much return to the functions of the skin before the wound event.

Examples of commercially available synthetic skin substitutes include Biobrane®, Dermagraft®, Integra®, Apligraf®, MatriDerm®, OrCel®, Hyalomatrix®, and Renoskin®.

U.S. Published Patent Application No. 2003/0059460 discloses a hybrid polymer skin substitute material comprising synthetic and natural polymers that can be used in regenerating living body tissue. The hybrid comprises a cross-linked naturally-occurring polymer and a biodegradation-absorbable synthetic polymer. A series of complicated process steps, however, must be undertaken to produce the hybrid material. In addition, the resulting hybrid material contains synthetic as well as naturally-occurring materials.

Most modern wound treatment products are so called wet-to-dry wound dressings that facilitate improved wound healing by keeping an appropriate moisture level on the wound. The products typically accumulate wound exudate and are exchanged at regular interval.

Biological skin substitutes may include, but are not limited to, skin grafts, including autologous skin grafts, syngeneic skin grafts, allogeneic skin grafts, xenogeneic skin grafts such as porcine skin grafts, cadaveric skin allografts, and amniotic tissue grafts.

Further, in recent years a new class of biological skin graft products has emerged that is intended to improve the micro milieu of the wound by providing the proliferating cells with shelter. Typically the new products are made from biologic materials containing intact collagen or reconstituted collagens. Examples include brands such as; Oasis, Matristem, Integra and Puracol. Those products are often referred to by clinicians as being matrix products. The matrix products are inserted into the wound where they are to attract cellular ingrowth. A secondary wet-to-dry wound dressing is then applied on top of the wound dressing.

One example of a matrix product derived from intact, decellularized fish skin is described in U.S. Pat. No. 8,613,957 B2, granted Dec. 24, 2013, and incorporated herein by reference in its entirety. The decellularized fish skin product describes by U.S. Pat. No. 8,613,957 serves as a scaffold material that provides an intact scaffold for support for ingrowth of endothelial and/or epithelial cells. The decellularized fish skin scaffold material is biocompatible thus can be integrated by the host. Omega3 Wound is a commercially available skin substitute made from the minimally processed skin of wild-caught Atlantic cod originating from Iceland. The fish skin is structurally alike to human skin with three basic layers including epidermis, dermis, and hypodermis and contains proteins, lipids, fatty acids, and other bioactive compounds that are homologous to human skin.

Examples of other biological skin substitutes include those described in U.S. Pat. No. 6,541,023, which describes the use of porous collagen gels derived from fish skin for use as tissue engineering scaffolds. Preparation of the collagen gels involves grinding the fish skin. Additionally, Chinese Patent No. 1068703 describes a process for preparing fish skin for dressing burn wounds, involving separating fish skin from the fish body and placing the skin in a preservation solution of iodine tincture, ethanol, borneol, sulfadiazine zinc and hydrochloric acid in amounts sufficient to establish a pH value of 2.5-3. However, these products can be difficult to handle as the product of U.S. Pat. No. 6,541,023 is in a gel form and the product of China Patent No. 1068703 is stored in a solution.

In addition, a number of extracellular matrix products for medical uses have been derived from human skin (ALLODERM® Regenerative Tissue Matrix (LifeCell)); fetal bovine dermis (PRIMATRIX™ Dermal Repair Scaffold (TEI Biosciences)); porcine urinary bladder (MATRISTEM™ Extracellular Matrix Wound Sheet (Medline Industries, Inc.)); and porcine small intestinal submucosa (OASIS® Wound Matrix (Healthpoint Ltd.)).

The group of skin substitutes which can be used as examples of skin substitutes according to the present disclosure is a large and varied. The AHRQ Technology Assessment Program entitled “Skin Substitutes for Treating Chronic Wounds” Technical Brief Project ID WNDT0818, published Feb. 2, 2020, which is incorporated herein by reference, in Table 2, on pages 9-13, identified 76 commercially available products with few studies comparing them internally. Each of these listed skin substitutes may be an embodiment of a skin substitute according to the present disclosure.

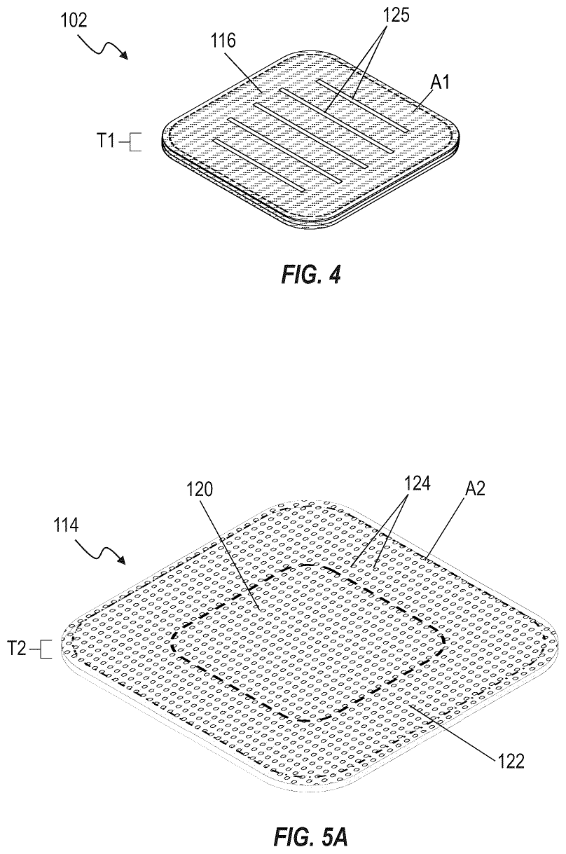

FIG. 4 depicts an exemplary skin substitute 102 according to the present disclosure. In an embodiment, the skin substitute 102 is an intact, decellularized fish skin product, such as one described in U.S. Pat. No. 8,613,957. The decellularized fish skin product serves as a scaffold material that provides an intact scaffold for supporting ingrowth of endothelial and/or epithelial cells. The decellularized fish skin product is capable of 3-D cell ingrowth ability due to its porous scaffold design. The decellularized fish skin product is biocompatible and can thus be integrated by the host at the wound site. In an embodiment, the skin substitute 102 is made from the minimally processed skin of wild-caught Atlantic cod originating from Iceland. The fish skin is structurally alike to human skin with three basic layers including epidermis, dermis, and hypodermis and contains proteins, lipids, fatty acids, and other bioactive compounds that are homologous to human skin.

The fish skin embodiment of the skin substitute 102 is preferably treated with one or more decellularizing solutions to remove cellular material, including antigenic material, from the fish skin with minimal to no damage to the mechanical and structural integrity and biological activity of the naturally occurring extracellular matrix. The terms “extracellular matrix” or “ECM” as used herein refer to the non-cellular tissue material present within the fish skin that provides structural support to the skin cells in addition to performing various other important functions. The ECM product described herein does not include matrix material that has been constituted or re-formed entirely from extracted, purified, or separated ECM components (e.g., collagen). According to one or more embodiments, the ECM product is in the form of particles, or a sheet, or a mesh.

The terms “acellular,” “decellularized,” “decellularized fish skin,” and the like as used herein refer to a fish skin product from which a substantial amount of cellular and nucleic acid content has been removed leaving a complex three-dimensional interstitial structure of ECM. Decellularization disrupts the cell membranes and releases cellular content. Decellularizing may involve one or more physical treatments, one or more chemical treatments, one or more enzymatic treatments, or any combination thereof. This material is cost effective so it can be converted into a usable resource and retains key biological features, comparable to other animal-derived skin technologies.

In certain embodiments, decellularization (and other optional processing steps) does not remove all of the naturally occurring lipids from the lipid layer of the fish skin. Thus, the scaffold material can comprise one or more lipids from the fish skin, particularly from the lipid layer of the fish skin. The lipids in the scaffold material can include, for example, fatty acyls (i.e., fatty acids, their conjugates, and derivatives). In certain embodiments the fatty acids include omega-3 fatty acids, such as eicosapentaenoic acid (EPA) and docosahexaenoic acid (DHA) (found in high concentration in fish oil). The omega-3 fatty acids enhance tissue regeneration and enhance the fish skin's ability to act as a bacterial barrier. The fish skin embodiment of the skin substitute 102 also provides the unique advantage of avoiding cultural and/or religious barriers to clinician and patient acceptance. Moreover, the fish skin embodiment of the skin substitute 102 is hypoallergenic, avoids risk of disease transfer, and does not require multi-layer grafts.

As shown in FIG. 2, the skin substitute 102 depicted features fenestrations 125 to facilitate wound exudate drainage during healing. The fenestrations 125 may be designed as slots, apertures, or other openings that allow proper drainage from the wound contacting surface 116 through the thickness T1 of the skin substitute 102. Providing fenestrations 125 as slots allow for flow of wound exudate through the skin substitute 102 and increases flexibility of the wound treatment device 100 while minimizing product loss and maximizing the contact area of the skin substitute 102 to the wound site. In an embodiment, the skin substitute 102 comprises between 1 and 10 linear fenestrations 125, preferably five linear fenestrations. The design of the fenestrations may be varied, depending on the size of the skin substitute or application of the wound treatment device 100.

In an embodiment, the skin substitute 102 is shaped like a square or in a generally squarish shape and has a surface area A1 between 2 square cm and 10 square cm. The skin substitute 102 may have a surface area A1 less than 2 square cm or greater than 10 square cm; however, it is preferred that the skin substitute 102 has a surface area A1 that is approximately equal to the wound size. The shape of the skin substitute may be of various shapes, including, but not limited to, a square, a circle, a triangle, or any other shape, symmetric of asymmetric, and or an irregular shape, or a shape that matches generally the shape of the wound. The size of skin substitute 102 is of course non-limiting, and may be produced or provided, or trimmed to fit the size and shape of the wound to be treated. Further, the skin substitute 102 can be configured as a meshed decellularized fish skin, particalized, comminuted, or otherwise processed into various sizes and shapes (square, rectangular, pentagonal, hexagonal, etc.). According to one or more embodiments, the extracellular matrix product of the skin substitute 102 is in the form of particles, a powder formed of extracellular matrix, a composite material including a skin substitute material, or a sheet, or a mesh.

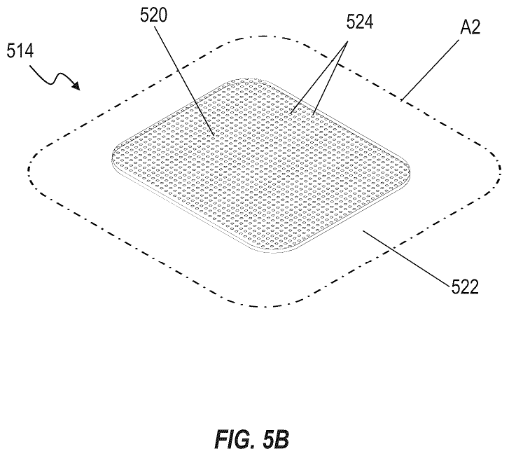

FIG. 5A depicts an exemplary polymer contact layer 114 according to the present disclosure. In an embodiment, the polymer contact layer 114 comprises a silicone layer adapted to be in contact with the skin substitute 102, or optionally the skin substitute 102 and the peri-wound skin 103. In an embodiment, the polymer contact layer 114 comprises a silicone contacting layer coated on a polyurethane membrane. This integrated, overlapping silicone contact layer with a polyurethane membrane holds the skin substitute 102 in place and helps protect both the granulating wound and the skin substitute 102 after an absorbent core 104 or other covering has been applied on top. In an embodiment, the polymer contact layer 114 is used as a non- or gentle-adherent wound contact layer. The polymer contact layer 114 allows for painless removal from the skin. In a preferred embodiment, the polymer contact layer 114 is non-adherent directly to the wound and exhibits soft adherence on peri-wound skin. The polymer contact layer 114 is configured to remain in place, e.g., fixed in the same location on the skin as originally applied, for up to fourteen days. In an embodiment, the period of time may be for up to fourteen days, the lifetime approved duration of the device, or may be for a duration that allows for cellular ingrowth and tissue regeneration at the wound site.

In an embodiment, the polymer contact layer 114 comprises various levels of adhesion. The polymer contact layer 114 may comprise a central portion 120 having a gentle adhesion between 0.015 and 0.85 N/cm, preferably between 0.035-0.8 N/cm, wherein the perimeter portion 122 of the polymer contact layer 114 exhibits a greater adhesion. In an alternative embodiment, the polymer contact layer 114 may comprise a perimeter portion 122 having a gentle adhesion between 0.015 and 0.85 N/cm, preferably between 0.035-0.8 N/cm, wherein the central portion 120 of the polymer contact layer 114 exhibits a greater adhesion.

In an embodiment, the polymer contact layer 114 comprises a plurality of apertures 124. The apertures 124 extend through and are continuously formed through the polymer contact layer 114. In an embodiment, the apertures 124 are provided as circular perforations to allow for wound exudate to flow freely from the wound site through the polymer contact layer 114. In an exemplary embodiment, the diameter of the apertures is between 0.5 mm and 5 mm, preferably between 1 mm and 4 mm.

The polymer contact layer 114 has a predetermined porosity to permit wound exudate to pass through the thickness of the polymer contact layer 114. The predetermined porosity defines a distribution of apertures 124 over a surface area A2 of the polymer contact layer 114. The distribution of apertures 124 is configured to provide sufficient transfer of wound exudate while allowing sufficient contact between the surface area of the silicone layer and the skin to maintain a gentle adherence. Preferably, distribution of the apertures 124 over the surface area of the polymer contact layer 114 is porous enough without compromising the sheet-like nature of the skin substitute 102. The apertures 124 are depicted as being formed over the entire surface area A2 of the polymer contact layer 114. However, in another embodiment of the polymer contact layer 514, as shown in FIG. 5B, the apertures 524 are confined only to a portion or limited portions of the polymer contact layer, such as, for example, the central portion 520 of the polymer contact layer 514 while apertures are not provided in another portion of the polymer contact layer, such as, for example, the perimeter portion of the polymer contact layer 514, for example, that are intended or configured to be arranged over the peri-wound skin.

In an embodiment, as shown for example in FIGS. 5A and 5B, the polymer contact layer 114, 514 is shaped like a square ad has a surface area A2 between 4 square cm and 20 square cm. The surface area A2 may be less than 4 square cm or greater than 20 square cm. It is preferred that the ratio of surface areas A2 to the surface area A1 of the skin substitute 102 is 2:1, 3:1, 3:2, 4:1, 4:3, 5:1, 5:2, 5:3, or 5:4. The ratios of the surfaces areas A2 to surface area A1 can be varied depending on the application; however, it is preferable that the surfaces area A2 is sufficiently greater than and sufficiently encompasses the surface area A1 so that the polymer contact layer 114 remains in place and in contact with the peri-wound skin for the desired duration.

In applications where the absorbent core 104 is intended to be removed from the skin substitute 102, silicone is a preferred material for the polymer contact layer 114 and is preferred over alternative adhesives and polymers because it avoids dermatitis and prevents microbial growth. Additionally, the biocompatible properties of the silicone demonstrate low thermal conductivity, low chemical reactivity, and low toxicity. In an embodiment, it is preferred that the polymer contact layer 114 is transparent for observing the peri-wound skin and passage of exudate through the corresponding apertures 124. In an alternative embodiment, the polymer contact layer 114 is colored or skin-toned to provide an aesthetically pleasing appearance or noticeable indication of the wound site.

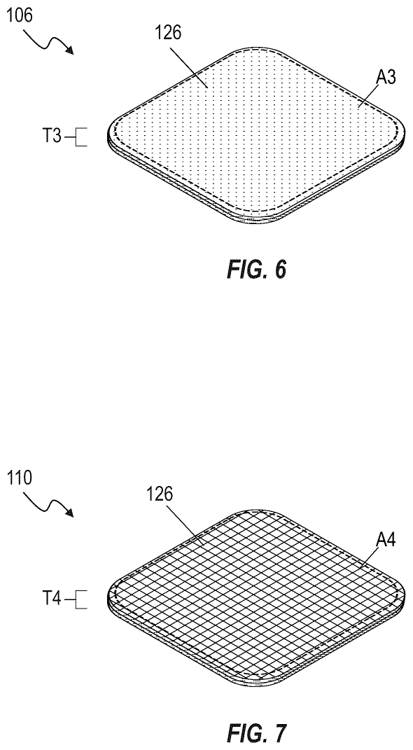

FIGS. 6 and 7 depicts aspects of the absorbent core 104. In a preferred embodiment, the absorbent core 104 in a preferred embodiment comprises a hydrophilic synthetic polymer conformable to body surfaces and adapted to be capable of absorbing fluid. It is desirable that the absorbent core 104 absorb exudate rapidly so as to enhance its effectiveness as a wound dressing, and in particular, the fluid uptake to the receptacles containing the absorbent material. In addition to absorption, an effective wicking mechanism is desirable, that is the absorbent core 104 should rapidly direct fluids away from the proximal surface of the absorbent core 104 to more remote areas for storage (i.e., the receptacles containing the discrete portions of absorbent material) that is more distant from the proximal surface, so as to minimize local saturation and maximize the efficiency of the absorbent core 104.

A preferable absorbent core 104 is constructed of flexible open-cell foam that is at least slightly hydrophilic. Suitable foams have an open cell size of 30 to 700 microns, and preferably a cell size of 50 to 300 microns. The open cells permit transport of fluid and cellular debris into and within the foam, and it preferred that the cell size of areas of the foam be of sufficient size to encourage capillary action and promote fluid transport.

The absorbent core 104 may expand about 135% of its size when saturated with fluid. The absorbent core 104 may expand to greater than 100% of its initial size when saturated with fluid up to 200% or great. In preferred embodiments, the absorbent core 104 expands to about 110% to about 170%. In more preferred embodiments the absorb core expands from about 125% to about 155% of its initial size. When combined with the facing and backing layer of the invention, the absorbent core 104 may expand to only about 110% of its dry size when exudate laden.

In accordance with one embodiment of the invention, the absorbent core 104 comprises a gradient of cell sizes across the thickness of the absorbent core 104 such that the cell size decreases in the direction of the distal surface and of the absorbent core 104. Since the cell sizes are greater at and near the proximal surface of the absorbent core 104, the capillary forces are stronger and therefore will drain fluid near the proximal surface of the absorbent core 104 and draw the fluid towards microcapillaries 126. In addition, the absorbent core 104 may include a cell size gradient that is directed towards the microcapillaries 126, thereby providing localized regions in the absorbent core 104 that are configured to have increased capillary forces directed towards the microcapillaries 126 to aid in the guidance of fluid thereto.

FIG. 6 depicts an exemplary conformable foam layer 106 of the absorbent core 104 that may be used in accordance with the disclosed wound treatment device 100. In an embodiment, the surface area A3 of the conformable foam layer 106 is substantially equal to the surface area A1 of the skin substitute A1. The conformable foam layer 106 is composed of a foam polymer having low resilience (i.e., memory foam capability) that leaves a temporary imprint in the foam after compression to provide superior comfort. The conformable foam layer 106 may be made, for example, from polyurethane, cellulose, carboxylated butadiene-styrene rubber, polyester foams, hydrophilic epoxy foams or polyacrylate. In a preferred embodiment, the conformable foam layer 106 is formed from hydrophilic polyurethane foam. Since the aforesaid foams are hydrophilic per se and further in view of the use of the microcapillaries and/or use of superabsorbent polymers, it is not necessary to treat the foams to render them more hydrophilic in a preferred embodiment.

The conformable foam layer 106 is viscoelastic and has adequate flexibility and thickness to conform to the shape of the wound bed after absorption of exudate. The conformable foam layer 106 allows for the wound treatment device 100 to conform into wound beds having depths between 0 and 10 cm, or between 0 and 5 cm, more preferably between 0 and 4 cm, or between 0 and 2 cm to match the shape of the wound and create an intimate fit to accommodate and couple to a concavity of the wound. In a preferred embodiment, the conformable foam layer 106 conforms into the wound bed up to a depth of 2 cm.

FIG. 7 depicts an exemplary receptacle layer 110 of the absorbent core 104 that may be used in accordance with the disclosed wound treatment device 100. In an embodiment, the receptable layer 110 is formed as a monolithic layer with the conformable foam layer 106, comprising the same material. Alternative embodiments provide the conformable foam layer 106 and receptacle layer 110 as being separate and distinct. Alternatively, the absorbent core 104 may comprise a multi-layer structure having more than two layers. For example, the absorbent core may be a multi-layer structure including alternating layers of a conformable foam layer 106 and a receptable layer 110. Or In an embodiment, the receptacle layer 110 of the absorbent core 104 comprises superabsorbent polymeric granulates, flakes, or powders that swell on exposure to water and form a hydrated gel (hydrogel) by absorbing large amounts of water. Superabsorbents are defined herein as materials that exhibit the ability to absorb large quantities of liquid, i.e., in excess of 10 to 15 parts of liquid per part thereof. These superabsorbent materials generally fall into three classes, namely starch graft copolymers, cross-linked carboxymethylcellulose derivatives and modified hydrophilic polyacrylates. Examples of such absorbent polymers are hydrolyzed starch-acrylonitrile graft copolymer, a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-crosslinking polyacrylic acid, a crosslinked polyacrylate salt, carboxylated cellulose, and a neutralized crosslinked isobutylene-maleic anhydride copolymer. Superabsorbent particulate hydrophilic polymers also are described in detail in U.S. Pat. No. 7,468,471, granted Dec. 23, 2008 and incorporated herein by reference. Preferably, the super absorbent particles used in the dressing of the present invention are preferably composed of cross-linked polyacrylic-acid.

The super absorbent particles are preferably in the form of granules or flakes to provide a greater available surface area hydrocolloid. The size of the super absorbent particles is typically within the range of 1 to 1000 micrometers when dry. Preferably, the particle size range of the absorbent particles is 100 to 900 micrometers. The particles which are insoluble in a wound environment have an absorptive capacity greater than 0.5 of water per gram of dry particles.

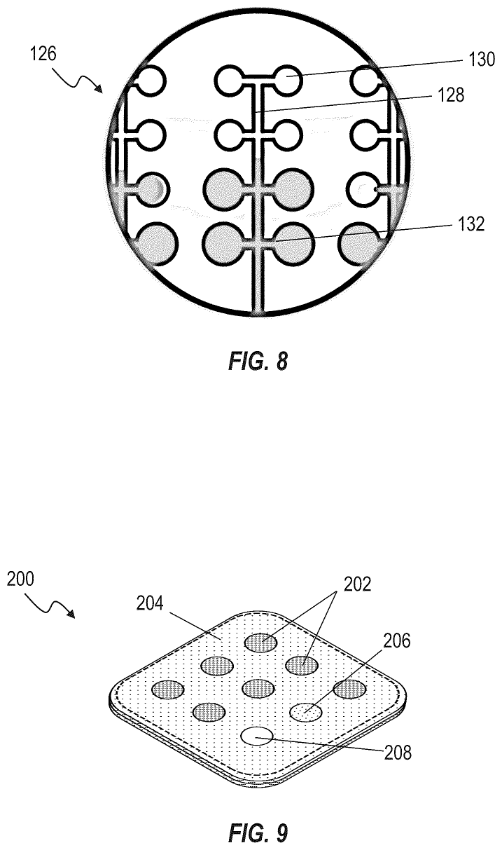

FIG. 8 depicts an exemplary configuration of a microcapillary 126 use in the absorbent core 104 of the disclosed wound treatment device 100. Each microcapillary is preferably formed as a channel 128 comprising lateral pockets 130 for containing fluid uptake or retaining exudate 132. As observed in FIG. 8, the lateral pockets 130 expand upon filling with exudate 132. In an embodiment, the microcapillaries 126 comprise superabsorbent particulate within the lateral pockets 130 to enhance fluid uptake.

FIG. 9 depicts an embodiment of the wound treatment device 200 according to the present disclosure comprises discrete, segmentized skin substitute portions 202 that are mechanically integrated into the absorbent core 204. In an embodiment, the skin substitute portions 202 are punched within partially formed cavities 206 and/or conduits 208 of the absorbent core 204. As the skin substitute portions 202 are mechanically integrated with the absorbent core 204, the wound treatment device 200 does not require a separate polymer contact layer. In an embodiment, the skin substitute portions 202 are formed with a skin substitute layer (e.g., 102), and is adapted with a LEGO-like fit to one or more cavities 206 or conduits 208 of the absorbent core 204. In an alternative embodiment, the discrete, segmentized skin substitute portions 202 are particlized and configured to be deposited into the wound bed before application of the absorbent core 204. Thus, with its conformable properties, the absorbent core 204 applies pressure to the particlized skin substitute portions 202 along the wound bed and prevents pooling of exudate.

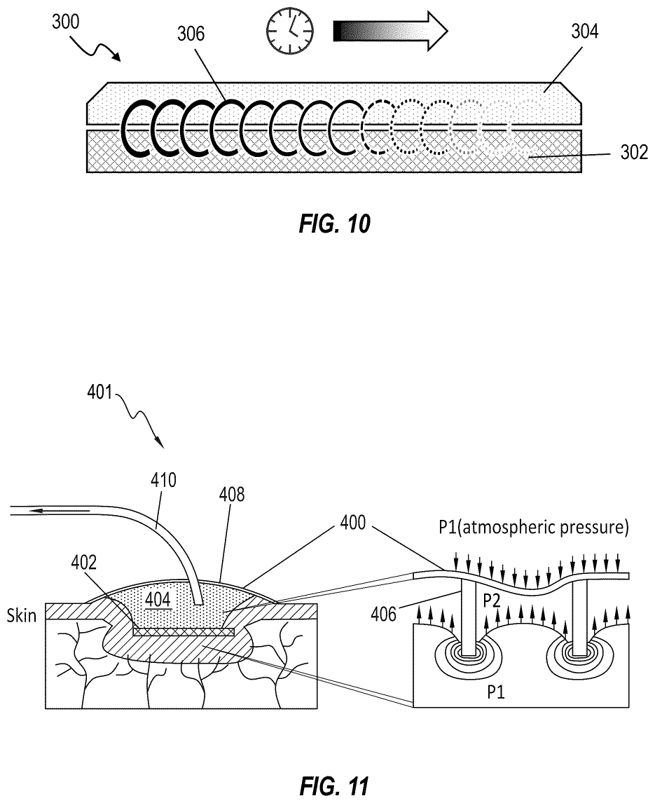

FIG. 10 depicts an embodiment of the wound treatment device 300 comprising an alternative means for attaching a skin substitute 302 to an absorbent core 304. The wound treatment device 300 comprises a suture 306 for combining the skin substitute 302 with the absorbent core 304. In an embodiment, the suture 306 is a biodegradable synthetic polymer that is biocompatible with the human body. The degradation rate of the biodegradable suture 306 can be customized to suit its application. After the suture 306 dissolves, the absorbent core 304 may be removed from the skin substitute 302, wherein the skin substitute preferably remains integrated with the wound site to facilitate tissue regeneration.

FIG. 11 depicts a negative pressure wound therapy (“NPWT”) application 401 comprising a wound treatment device 400 of the present disclosure. The wound treatment device 400 shown in FIG. 11 with the negative pressure wound therapy may be any of the wound treatment devices as shown and described herein. The NPWT application 401 continuously or intermittently apply sub-atmospheric pressure to the surface of a wound. The wound treatment device comprises a skin substitute 402 and an absorbent core 404. The wound treatment device 400 is connected to single or multi lumen tube or conduit 410 that is operatively connected with a pump assembly. The conduit 410 is integrated with the occlusive wound treatment device 400 and joined with the absorbent core 404. The wound contacting surface (i.e., skin substitute 402) of the wound treatment device 400 is porous to allow for an even distribution of pressure throughout the wound. The wound treatment device 400 dressing is placed in direct contact with the wound, and the NPWT application 401 features a drain attached to a vacuum device. The wound treatment device 400 comprises a backing layer 408 formed with an air-tight material. In a preferred embodiment, the backing layer 408 comprises polyurethane and extends approximately 2 cm beyond the wound edges to provide an airtight seal. In a preferred embodiment, the wound treatment device 400 comprises discrete, incompressible struts 406 to create a continuous tension network to refine the shape of the wound treatment device 400 and create a uniform distribution of pressure across the wound bed. The struts 406 synergistically facilitate vertical absorption of exudate along with the absorbent core 404 and negative pressure supplied by the NPWT application 401.

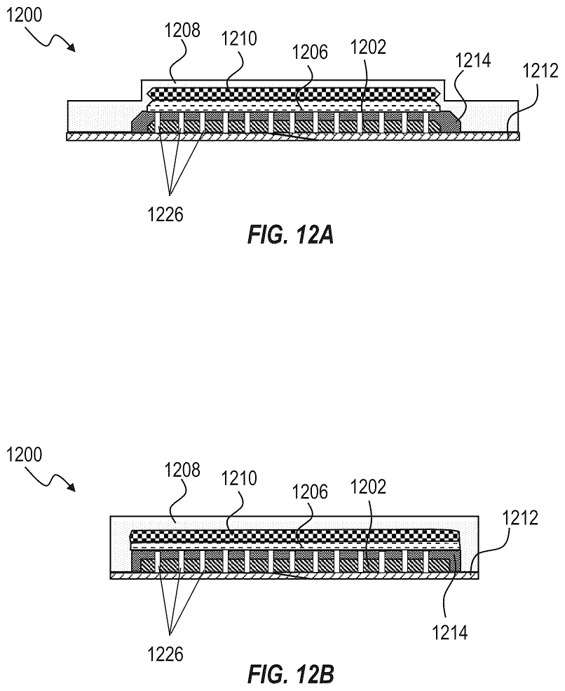

FIGS. 12A and 12B depict schematic wound treatment devices 1200 according to further, similar embodiments. In the embodiment of both FIGS. 12A and 12B, the wound treatment device 1200 is a wound dressing comprising an intact skin substitute 1202 used to support tissue regeneration of wounds. Similar to the would treatment device as shown in the embodiment of FIGS. 1 and 2, the wound treatment device 1200 is suitable for management of the following wounds: partial and full thickness wounds, pressure ulcers, venous ulcers, chronic vascular ulcers, diabetic ulcers, trauma wounds (e.g., abrasions, lacerations, partial thickness burns, skin tears), and surgical wounds (e.g., donor sites/grafts, post-Mohs surgery, post-laser surgery, podiatric, wound dehiscence).

The wound treatment device 1200 of each of FIGS. 12A and 12B comprises a skin substitute 1202 and an absorbent core 1204, the skin substitute 1202 being coupled to the absorbent core 1204. The skin substitute 1202 is adhered to a polymer contact layer 1214 that is disposed between the skin substitute 1202 and a absorbent core 1204. The absorbent core 1204 comprises a conformable foam layer 1206 and a receptacle layer 1210 for absorbing and containing wound exudate. The wound treatment device 100 includes a backing layer 1208 to protect the wound bed and peri-wound skin and also to protect the lower layers from damage and contamination. The wound treatment device 1200 also comprises a release liner 1212 for aseptic handling of the wound treatment device 100 before application at the wound site.

In the embodiments of FIGS. 12A and 12B, the polymer contact layer 1214 and skin substitute 1202 comprise a plurality of apertures 1226 that extend through and align in both the polymer contact layer 1214 and the skin substitute 1202. The apertures 1226 extend through and are continuously formed through the polymer contact layer 1214 and the skin substitute 1202. All or some of the apertures that extend through the polymer contact layer 1214 extend through and align with all or some of the apertures of the skin substitute 1202. In an embodiment, the apertures 1226 are provided as circular perforations to allow for wound exudate to flow freely from the wound site through the polymer contact layer 1214. In an exemplary embodiment, the diameter of the apertures is between 0.5 mm and 5 mm, preferably between 1 mm and 4 mm.

The embodiments of FIGS. 12A and 12B are composed of similar materials and generally similar structures, but the embodiments of FIGS. 12A and 12B differ from each other in the relative size or diameter of the skin substitute 1202, the polymer contact layer 1214, and the layers of the absorbent core 1204. In a preferred embodiment, as shown in FIG. 12B, the polymer contact layer 1214 is generally the same size or close to the same size in terms of width or diameter as the layers of the absorbent core 1204, including similar in size, the same, or close to the same size of width or diameter as the receptacle layer 1210 and the conformable foam layer 1206. In an even more preferred embodiment, the polymer contact layer 1214 is similar in size as the layers of the absorbent core 1204 but the polymer contact layer 1214 is just slightly larger/greater than the layers of the absorbent core 1204 to prevent possible contact at the edges between the skin substitute 1202 and the absorbent core 1204 after being placed on the wound and absorption is made which changes the size of the area of the absorbent core 1204. In an even preferred embodiment, the conformable layer 1206 is slight layer in area than the receptacle layer 1210 of the absorbent core 1204. But, as shown in the embodiment, the relative widths and/or diameters of the polymer contact layer 1214, the skin substitute 1202, and the different layers of the absorbent core 1204, such as the receptacle layer 1210 and the conformable foam layer 1206 may vary relative to each other to achieve desired function. And the shown relative sizes should be understood to be non-limiting.

Similarly, in the embodiments shown in here, including those of FIGS. 1 and 2, and 12A and 12B should be understood to be schematic representations and should not be necessarily understood to show relative thickness of the different layers of the wound treatment device. In a preferred embodiment, the thickness of the absorbent core 1204 may be set or determined based on the desired functions and absorbing characteristics of the wound treatment device. In an embodiment, the absorbent core 1204 may initially (before absorbing liquid) between 0.01 and 500 mm, between 0.1 and 100 mm, between 0.2 and 50 mm, between 0.3 and 30 mm, or between 0.4 and 20 mm. In a more preferred embodiment, the thickness of the absorbent core is between 1 and 10 mm. Further, the thickness of the different layers of the absorbent core 1204 may vary. For example, the thickness of the conformable foam layer 1206 may be between 0.01 and 300 mm, between 0.1 and 100 mm, between 0.2 and 50 mm, between 0.3 and 30 mm, between 0.4 and 20 mm, between 0.5 and 10 mm. In a preferred embodiment, the thickness of the conformable foam layer 1206 is about 1 to 5 mm, and most preferably between 1.5 and 4 mm, and even more preferably between about 2 mm to 3 mm, and preferably about 2.3 mm. The thickness of the receptacle layer 1210 may also be between 0.01 and 300 mm, between 0.1 and 100 mm, between 0.2 and 50 mm, between 0.3 and 30 mm, between 0.4 and 20 mm, between 0.5 and 10 mm. In a preferred embodiment, the thickness of the receptacle layer 1210 is about 1 to 5 mm, and most preferably between 1.5 and 5 mm, and even more preferably between about 2 mm to 5 mm, and even more preferably between 2.5 and 4.5 mm, and most preferably about 3.5 mm. The thickness of the layers of the absorbent core 104 of FIGS. 1 and 2 may share similar ranges and quantities as those provided for the embodiments of FIGS. 12A and 12B.

The skin substitute 1202 has a first thickness T1. Generally, the first thickness is from about 0.1 to 10 mm thick, preferably about 0.1 to 4.0 mm thick (i.e., in cross-section), such as 0.5, 1.0, 1.5, 2.0, 2.5, 3.0 or 3.5 mm thick. The thickness can depend on a number of factors, including the type of skin substitute, and in the case that the skin substitute is a fish skin substitute, the thickness depends on the species of fish used as the starting material, processing, lyophilization, and/or rehydration, as discussed in greater detail in U.S. Pat. No. 8,613,957. In an embodiment, the skin substitute 1202 has multiple layers. In one embodiment, the thickness is uniform across the surface area A1 of the skin substitute 1202. In an alternative embodiment, the first thickness may taper at the edge(s) defining the surface area A1 of the skin substitute 1202.

The polymer contact layer 1214 also has a thickness. The combined thickness the polymer contact layer 1214, that is, of all layers comprised by the contact layer 1204 n the case that the contact layer is a multi-layer structure or a multi-layer film, is preferably greater than 0.2 mm. Generally, the thickness of the contact layer 1204 is from about 50 to 200 gsm, such as 100, 110, 120, 130, 140, 150, 160, 170, 180, or 190 gsm thick. “GSM” or Grams per Square Meter is a unit for measuring the thickness of materials like paper and fabric. The gsm thickness is dependent on both the weight of the silicone gel used in a silicone layer of the contact layer and the surface area A2 of the silicone layer. In an embodiment, the second thickness of the contact layer is uniform across the surface area A2 of the contact layer 1214. In an alternative embodiment, the second thickness of the contact layer 1214 tapers at the edge(s) defining the surface area A2 of the silicone layer 106. In an embodiment, the second thickness T2 is reduced at the central portion of the polymer contact layer 1214 to receive and partially contain the skin substitute 1202 within a recess. Generally, the thickness of the contact layer permits the contact layer to be both sufficiently pliable and elastic to allow it to conform, with the skin substitute to the concavity of the wound bed. The contact layer preferably also exhibits both sturdiness and strong resistance to humidity and organic solvents.

The polymer contact layer 1214 and the skin substitute 1202 has a predetermined porosity to permit wound exudate to pass through the thickness of the polymer contact layer 1214. The predetermined porosity defines a distribution of apertures 1226 over a surface area of the polymer contact layer 1214 and the skin substitute 1202. The distribution of apertures 1226 is configured to provide sufficient transfer of wound exudate while allowing sufficient contact between the surface area of the silicone layer and the skin to maintain a gentle adherence. Preferably, distribution of the apertures 1226 over the surface area of the polymer contact layer 1214 and the skin substitute 1202 is porous enough without compromising the sheet-like nature of the skin substitute 1202 or the integrity of the skin substitute 1202. The apertures 1226 are depicted as being formed over the entire surface area of the polymer contact layer 1214 and the skin substitute 1202. However, in another embodiment of the polymer contact layer, the apertures are confined only to a portion or limited portions of the polymer contact layer, such as, for example, the central portion of the polymer contact layer while apertures are not provided in another portion of the polymer contact layer, such as, for example, the perimeter portion of the polymer contact layer, for example, that are intended or configured to be arranged over the peri-wound skin.

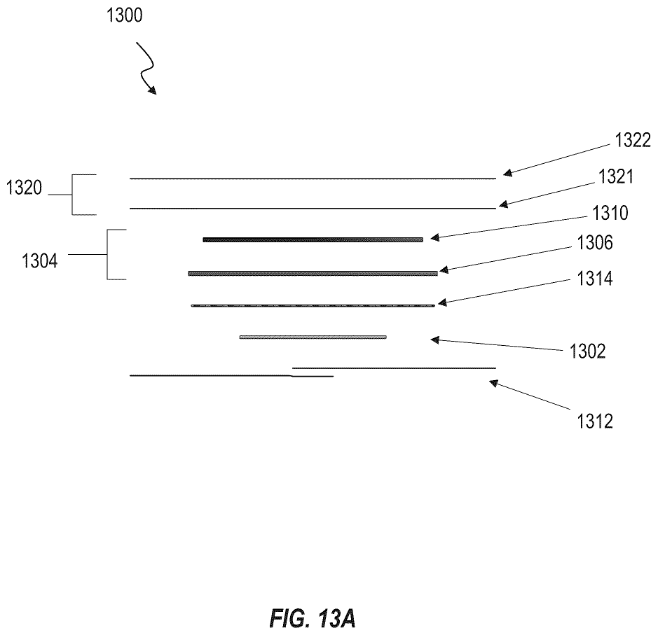

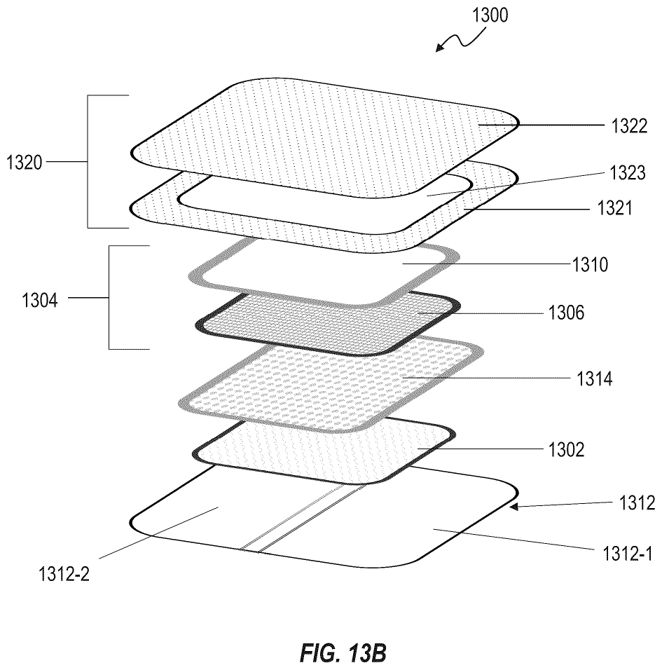

FIGS. 13A and 13B each depict a schematic of a wound treatment device 1300 according to another embodiment. In the embodiment of FIGS. 13A and 13B, the wound treatment device 1300 is a wound dressing comprising an intact skin substitute 1302 used to support tissue regeneration, and particularly skin regeneration, in a wound.

The wound treatment device 1300 comprises a skin substitute 1302 and an absorbent core 1304. The skin substitute 1302 adheres to a contact film 1314 that is disposed between the skin substitute 1302 and absorbent core 1304. The absorbent core 1304 comprises a conformable foam layer 1306 and an absorbent layer 1310 for absorbing and containing wound exudate. The wound treatment device 1300 includes a backing layer 1320 as a backing layer or top layer or top film that is provided to protect the lower layers (1302, 1314, 1304) from damage and contamination and also to protect the wound bed 1301 and peri-wound skin 1303. The backing layer 1320 is preferably a multi-layer film including, in a preferred embodiment, an outer layer 1322 that overlies an inner layer 1321. The wound treatment device 1300 also comprises a release liner 1312 for aseptic handling of the wound treatment device 1300 before application at the wound site.

In the embodiment of FIGS. 13A and 13B, the skin substitute 1302 has a smaller surface area than the contact film 1314, such that the contact film 1314 extends beyond the edge of the skin substitute 1302. In the embodiment of FIGS. 13A and 13B, the absorbent core 1304, including the absorbent layer 1310 arranged overlaying the conformable foam layer 1306 are sized generally similarly in surface area to the contact film 1314. Preferably, the absorbent layer 1310 has a slightly smaller area than the conformable foam layer 1306, to prevent contact between the absorbent layer 1310 and the peri-wound skin 1303. And in this embodiment, it is preferable that the backing layer 1320, including the outer layer 1322 and inner layer 1321. Preferably, the edges of the perimeter of both the outer layer 1322 and the inner layer 1321 are aligned, such that the area spanned by the outer layer 1322 and the inner layer 1321 are the same or substantially the same. Further, the area spanned by the backing layer 1320 and the release liner 1312 are similar and preferably aligned, the area of each of the backing layer 1320 and the release liner 1312 being larger than the interior layers (skin substitute 1302, contact film 1314, and absorbent core 1304), such that the interior surface of the release liner 1312 contacts the lower, interior surface of inner layer 1321 of the backing layer 1320.

As shown in FIGS. 13A and 13B, the release liner 1312 preferably includes two portions 1312-1, 1312-2, which meet and make contact with each other, preferably but not necessarily at or near the center, wherein the first portion 1312-1 maybe overlapped by the second portion 1312-2 of the release liner 1312, and the second portion 1312-2 may be folded outwardly to facilitate a gripping by a medical practitioner when applying the wound treatment device 1300. Or a the first portion 1312-1 may be a shorter component that is folded and the second portion 1312-2 may be a longer component that overlaps the shorter first portion 1312-1 for easy release. Both portions of the release liner 1312 may be made of a plastic material, a polymer material, or, for example, a thermoplastic. In a preferred embodiment, the release liner is made of low-density polyethylene (LDPE). Such a LDPE preferably has a density range of 917-930 km/m³. Preferably the material of the release liner 1312 is resistant to impact, is flexible, resistant to moisture and provides good chemical resistance. In other embodiments, the release layer 1312 may be made of or include other polyethylene materials, such as high-density polyethylene (HDPE) and linear low-density polyethylene (LLPDE), polypropylene (PP), polyolefin, and/or polyethylene terephthalate (PET). According to another embodiment, the release liner 1312 may be made of or include a thin layer of polyvinyl chloride (PVC) and/or polyethylene terephthalate glycol (PETG). The release liner 1312 may be a single-layer structure or may be a multilayer structure.

In a preferred embodiment, the skin substitute 1302 is an extracellular scaffold, matrix product derived from intact, decellularized fish skin is described in U.S. Pat. No. 8,613,957 B2, granted Dec. 24, 2013, and incorporated herein by reference in its entirety. The decellularized fish skin product describes by U.S. Pat. No. 8,613,957 serves as a scaffold material that provides an intact scaffold for support for ingrowth of endothelial and/or epithelial cells. The decellularized fish skin scaffold material is biocompatible thus can be integrated by the host. Omega3 Wound is a commercially available skin substitute made from the minimally processed skin of wild-caught Atlantic cod originating from Iceland. The fish skin is structurally alike to human skin with three basic layers including epidermis, dermis, and hypodermis and contains proteins, lipids, fatty acids, and other bioactive compounds that are homologous to human skin. However, in the embodiment of FIGS. 13A and 13B, the skin substitute 1302 is not limited to an extracellular scaffold, matrix product derived from intact, decellularized fish skin, and may include or be comprised of other various skin substitutes as alternatively described above.

The skin substitute 1302 depicted in FIGS. 13B, similar to the skin substitute shown in FIG. 4, may include fenestrations 118 to facilitate wound exudate drainage during healing. The fenestrations 125 may be designed as slots, apertures, or other openings that allow proper drainage from the wound contacting surface 116 through the thickness T1 of the skin substitute 1302. Providing fenestrations 125 as slots allow for flow of wound exudate through the skin substitute 102 and increases flexibility of the wound treatment device 1300 while minimizing product loss and maximizing the contact area of the skin substitute 102 to the wound site. In an embodiment, the skin substitute 102 comprises between 1 and 10 linear fenestrations 125, preferably five linear fenestrations. The design of the fenestrations may be varied, depending on the size of the skin substitute or application of the wound treatment device 100. Or additionally or alternatively, the skin substitute 1302 may have small apertures, for example, round apertures, provided therein that similarly allow proper drainer from the wound through the thickness of the skin substitute 1302.

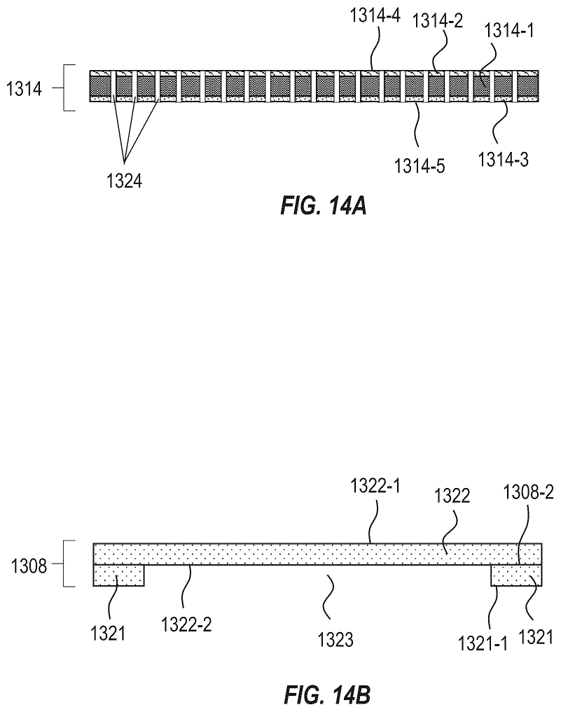

In a preferred embodiment, the contact film 1314 is a multi-layer structure. As shown, for example, in FIG. 14A, the contact film 1314 includes a core layer 1314-1 arranged between a top, first outer layer 1314-2 and a bottom, second outer layer 1314-3. In a preferred embodiment, the core layer 1314-1 is made of made of a plastic material, a polymer material, or, for example, a thermoplastic. In a preferred embodiment, the core layer 1314-1 of the contact film 1314 is made of polyethylene terephthalate (PET). Preferably the material of the core layer 1314-1 of the contact film 1314 is resistant to impact, is flexible, resistant to moisture and provides good chemical resistance. In other embodiments, the core layer 1314-1 of the contact film 1314 may be made of or include other polyethylene materials, such as high-density polyethylene (HDPE) and linear low-density polyethylene (LLPDE), polypropylene (PP), and/or is made of low-density polyethylene (LDPE). According to another embodiment, the core layer 1314-1 of the contact film 1314 may be made of or include a thin layer of polyvinyl chloride (PVC) and/or polyethylene terephthalate glycol (PETG). The core layer 1314-1 of the contact film 1314 itself may be a single-layer structure or may be a multilayer structure.

As shown in FIG. 14A, the core layer 1314-1 of the contact film 1314 preferably has a silicone adhesion provided thereon as the top layer 1314-2 and the bottom layer 1314-3. The silicone adhesion provided as the top payer 1314-2 and the bottom layer 1314-3 may be the same type of silicone adhesion or may be different silicone adhesion materials. The top surface 1314-4 of the top layer 1314-2 is configured to contact the bottom surface of the conformable foam layer 1306. And the bottom surface 1314-5 of the bottom layer 1314-3 is configured to contact the top surface of the skin substitute 1302.

The skin substitute 1302 is in direct contact with a contact film 1314 at the silicone adhesive layer 1314-3, which is a mildly cohesive silicone. The wound treatment device 1300 is thus configured to be applied to a wound bed 1301 such that the skin substitute 1302 is in contact with the wound bed 1301 and the contact film 1314 is layered between the skin substitute 1302 and absorbent core 1304. The contact film 1314 is integrally bonded to the absorbent core 1304 and removably adhered to the skin substitute 1302. The silicone adhesive bottom layer 1314-3 is advantageously in direct contact with the skin substitute 1302 material and is biocompatible with the skin. Additionally, the contact film preferably prevents exudate from transporting from the absorbent core 1304 back into the wound bed 1301, under compression. In an embodiment, the silicone layer 1314-3 provides a gentle adhering bond to the skin substitute 1302 for proper fixation and painless separation after healing has occurred. The silicone layer has sufficient tackiness to negate a need for an adhesive between the silicone layer and the skin graft material. An exemplary polymer contact layer for the silicone adhesive of the bottom layer 1314-3 is described in greater detail in U.S. Provisional Application No. 63/541,661, filed on Sep. 29, 2023 entitled “Wound Dressing”, which is incorporated herein by reference in its entirety.

In a preferred embodiment, the silicone adhesive layer 1312-3 has sufficient tackiness to negate a need for an additional adhesive between the silicone layer 1312-3 and the skin substitute 1302. Preferably, according to an embodiment, the contact film, comprising the silicone layer 1314-3 and core layer 1314-1, is hydrophobic and impermeable to liquid, to allow for complete immersion of the wound treatment within a saline solution before application to the skin. Advantageously, the silicone layer 1314-3 retains its gentle adhesive properties after immersion. After application of the skin graft material 1302 to a wound, after a period of time has elapsed, the silicone layer 1314-3 of the film layer 1314 is configured to conveniently separate from the skin substitute. In an embodiment, the period of time may be for up to fourteen days or may be for a time window that allows for cellular ingrowth and tissue regeneration at the wound site. During removal of the contact film 1314, an acellular dermal matrix of the skin substitute can remain on or integrated in the wound and can be absorbed by the wound during healing and will not be disturbed or damaged upon ultimate removal of the contact film 1314 from the skin substitute 1302. for the silicone adhesive layer 1314-3, sufficient tackiness may be defined as an adhesion between 0.015 and 0.85 N/cm, preferably between 0.035-0.8 N/cm.

Similarly, the top layer 1314-2 of the contact film 1314 is an adhesive layer, preferably a silicone adhesive layer that provides sufficient tackiness to permit the contact film 1314 to be separable from the bottom surface of the conformable foam layer 1306 such that the absorbent core 1304 may be separated from the top layer 1314-2 of the contact film 1314 without separating the bottom layer 1314-3 of the contact film 1314 from the skin substitute 1302. Accordingly, after the absorbent core 1304, and particularly the absorbent layer 1310 of the absorbent core 1304 is saturated or is otherwise desired to be replaced, the upper layers (the absorbent core 1304 and the backing layer 1320) may be removed and replaced with a new set of upper layers (the absorbent core 1304′ and the backing layer 1320′) as will be described hereinbelow.

The contact film 1314 has a predetermined porosity to permit wound exudate to pass through the thickness of the contact film 114. The predetermined porosity defines a distribution of apertures 1324 over a surface area A2 of the polymer contact layer 1314. The distribution of apertures 1324 is configured to provide sufficient transfer of wound exudate while allowing sufficient contact between the surface area of the silicone adhesive layer 1314-3 and the skin substitute to maintain a gentle adherence and sufficient contact between the top silicone adhesive layer 1314-2 and the absorbent core 1304. Preferably, distribution of the apertures 1324 over the surface area of the polymer contact layer 1314 is porous enough without compromising the sheet-like nature of the skin substitute 1302. The apertures 1324 are depicted as being formed over the entire surface area A2 of the polymer contact layer 114. However, similar to the embodiment shown in FIG. 5B, the apertures 1324 may be confined only to a portion or limited portions of the polymer contact layer, such as, for example, the central portion of the contact film 1314 while apertures are not provided in another portion of the polymer contact layer, such as, for example, the perimeter portion of the polymer contact layer 1314, for example, that are intended or configured to be arranged over the peri-wound skin.

The absorbent core 1304 comprises a conformable foam layer 1306 and an absorbent layer 1310 for absorbing and containing wound exudate. In a preferred embodiment, the conformable foam layer 1306 is configured to absorb exudate from the wound and provide a three-dimensional foam structure that confirms into the wound and effectively applies pressure to push excessive exudate out of the wound, causing the exudate to pass through the apertures 1324 of the contact film 1304, similar to as shown in the embodiment of FIG. 3. In a preferred embodiment, the absorbent layer 1310 is a super absorbent layer flued to the top surface of the conformable foam layer 1306 with permanent pressure sensitive hot melt (PSA) or holt melt glue. The PSA hot melt may include a blend of synthetic rubber, acrylic, or silicone polymers, combined with tackifiers and stabilizers to create a pressure-sensitive adhesive. Other forms of adhesives or glues may also be used to couple the absorbent layer 1310 to the conformable foam layer 1306. The absorbent layer 1310 locks away exudate received from the wound. In a preferred embodiment, the surface area of the absorbent layer 1310 is smaller than the surface area of the conformable foam layer 1306 to ensure that edges are sealed when the wound treatment device 1300 is assembled with the backing layer 1320 to prevent contact between the absorbent layer 1310 and the peri-wound skin 1303.

In a preferred embodiment, the absorbent core 1304 comprises preferably a hydrophilic synthetic polymer conformable to body surfaces and adapted to be capable of absorbing fluid. It is desirable that the absorbent core 1304 absorb exudate rapidly so as to enhance its effectiveness in the wound treatment device, and in particular, the fluid uptake to the receptacles containing the absorbent material. In addition to absorption, an effective wicking mechanism is desirable, that is the absorbent core 1304 should rapidly direct fluids away from the lower surface of the absorbent core 1304 to more remote areas for storage (i.e., the receptacles containing the discrete portions of absorbent material), so as to minimize local saturation and maximize the efficiency of the absorbent core 1304.

A preferable absorbent core 1304 is constructed of flexible open-cell foam that is at least slightly hydrophilic. Suitable foams have an open cell size of 30 to 700 microns, and preferably a cell size of 50 to 300 microns. The open cells permit transport of fluid and cellular debris into and within the foam, and it preferred that the cell size of areas of the foam be of sufficient size to encourage capillary action and promote fluid transport.

The absorbent core 1304 may expand about 135% of its size when saturated with fluid. When combined and restricted with the top layer of the wound treatment device, the absorbent core 104 may expand to only about 130%, to 120%, or 110% of its dry size when exudate laden.

According to an embodiment, the absorbent core 1304 comprises a gradient of cell sizes across the thickness of the absorbent core 1304 such that the cell size decreases in the direction of the distal surface and of the absorbent core 1304. Since the cell sizes are greater at and near the proximal surface of the absorbent core 1304, the capillary forces are stronger and therefore will drain fluid near the proximal surface of the absorbent core 1304 and draw the fluid towards microcapillaries. In addition, the absorbent core 1304 may include a cell size gradient that is directed towards the microcapillaries, thereby providing localized regions in the absorbent core 1304 that are configured to have increased capillary forces directed towards the microcapillaries to aid in the guidance of fluid thereto.

The conformable foam layer 1306 according to an embodiment is composed of a foam polymer having low resilience (i.e., memory foam capability) that leaves a temporary imprint in the foam after compression to provide superior comfort. The conformable foam layer 1306 may be made, for example, from polyurethane, cellulose, carboxylated butadiene-styrene rubber, polyester foams, hydrophilic epoxy foams or polyacrylate. In a preferred embodiment, the conformable foam layer 1306 is formed from hydrophilic polyurethane foam. Since the aforesaid foams are hydrophilic per se and further in view of the use of the microcapillaries and/or use of superabsorbent polymers, it is not necessary to treat the foams to render them more hydrophilic in a preferred embodiment.

The conformable foam layer 1306 in an embodiment is viscoelastic and has adequate flexibility and thickness to conform to the shape of the wound bed after absorption of exudate. The conformable foam layer 1306 allows for the wound treatment device 1300 to conform into wound beds having depths between 0 and 4 cm to match the shape of the wound and create an intimate fit. In a preferred embodiment, the conformable foam layer 1306 conforms into the wound bed up to a depth of 2 cm.

In an embodiment, the absorbent layer 110 is formed as a monolithic layer with the conformable foam layer 1306, and may, in some embodiments, comprise the same material. Alternative embodiments provide the conformable foam layer 1306 and receptacle layer 1310 as being separate and distinct. In an embodiment, the receptacle layer 1310 of the absorbent core 1304 comprises superabsorbent polymeric granulates, flakes, or powders that swell on exposure to water and form a hydrated gel (hydrogel) by absorbing large amounts of water. Superabsorbents are defined herein as materials that exhibit the ability to absorb large quantities of liquid, i.e., in excess of 10 to 15 parts of liquid per part thereof. These superabsorbent materials generally fall into three classes, namely starch graft copolymers, cross-linked carboxymethylcellulose derivatives and modified hydrophilic polyacrylates. Examples of such absorbent polymers are hydrolyzed starch-acrylonitrile graft copolymer, a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-crosslinking polyacrylic acid, a crosslinked polyacrylate salt, carboxylated cellulose, and a neutralized crosslinked isobutylene-maleic anhydride copolymer. Superabsorbent particulate hydrophilic polymers also are described in detail in U.S. Pat. No. 7,468,471, granted Dec. 23, 2008 and incorporated herein by reference. Preferably, the super absorbent particles used in the dressing of the present invention are preferably composed of cross-linked polyacrylic-acid.

The super absorbent particles are preferably in the form of granules or flakes to provide a greater available surface area hydrocolloid. The size of the super absorbent particles is typically within the range of 1 to 1000 micrometers when dry. Preferably, the particle size range of the absorbent particles is 100 to 900 micrometers. The particles which are insoluble in a wound environment have an absorptive capacity greater than 0.5 of water per gram of dry particles.

As shown in FIGS. 13A and 13B, and FIG. 14B, the wound treatment device 1300 includes a backing layer 1320 as a backing layer to protect the lower layers (1302, 1314, 1304) from damage and contamination and also to protect the wound bed 1301 and peri-wound skin 1303. The backing layer 1320 is preferably a multi-layer film including, in a preferred embodiment, an outer layer 1322 that overlies an inner layer 1321. The inner layer 1321 defines an inner, unoccupied space 1323 such that the backing layer 1320 is provided with a concavity at space 1323. In other words, the inner layer 1321 has a central portion removed at space 1321 such that only the perimeter portion remains. The top surface of the absorbent layer 1310 thus contacts the lower, inner surface 1322-2 of the outer layer 1322 through the space 1323 defined by the inner layer 1321. A silicone adhesive may be applied to the lower surface 1321-1 of the inner layer 1321, which bones to the skin, preferably the peri-wound skin of the patient when the wound treatment device 1300 is applied to the wound of the patient.

The outer layer 1322 in a preferred embodiment is a breathable film, preferably a breathable plastic film, more preferably a thermoplastic polyurethane (PU) film. The outer layer 1322 in a preferred embodiment is adhered to the upper surface of the inner layer 1321 with an acrylic adhesive or another adhesive at surface 1308-2. In a preferred embodiment, the adhesion of the outer layer 1322 to the upper surface of the inner layer 1321 at surface 1308-2 and the top surface of the absorbent layer 1310 at surface 1322-2 is sufficiently strong to ensure that the foam core 1304, including the conformable foam layer 1306 and the absorbent layer 1310 stays with the outer film 1322 of the backing layer 1320 when removed from the patient after treatment. In a preferred embodiment, the inner layer 1321 is also a breathable film, preferably a breathable plastic film, more preferably a thermoplastic polyurethane (PU) film, that is, preferably not in contact with the absorbent layer 1310 of the conformable foam layer 1306. It is noted that the outer layer 1322 and the inner layer 1321 may be of other breathable film materials. For example, these films may be made of, or include a combination of film materials, such as, a breathable thermoplastic copolyester (TPC), such as Arnitel®, a breathable polyethylene, such as Lumicene® M4040 and Supertough® 33ST22, or Sof-Flex™, or other films, not limited only to plastic or polymer-based films, but other bio-based films, or other types of films that are permeable to water vapor but impermeable to liquid. In a preferred embodiment, the material of the outer layer 1322 has sufficient elasticity so as to be able to stretch to accommodate the expansion of the absorbent core 1304, and particularly the absorbable receptacle layer 1310.

Although shown schematically in the drawings, the thickness of the backing layer 1320 may vary and should not be understood from the drawings to necessarily have a certain thickness relative to the other layers of the device. The thickness of the backing layer 1320 may be between 0.01 and 1000 μm, between 0.1 and 500 μm, between 0.2 and 200 μm, between 0.3 and 100 μm, between 5 and 90 μm, between 10 and 80 μm. In a preferred embodiment, the thickness of the outer layer 1322 is about 1 to 100 μm, and most preferably between 10 and 60 mm, and even more preferably between about 20 μm to 50 μm, and preferably about 30 μm. In a preferred embodiment, the thickness of the inner layer 1321 with the acrylic adhesive is about 1 to 100 μm, and most preferably between 10 and 60 mm, and even more preferably between about 20 μm to 50 μm, and preferably about 45 μm.

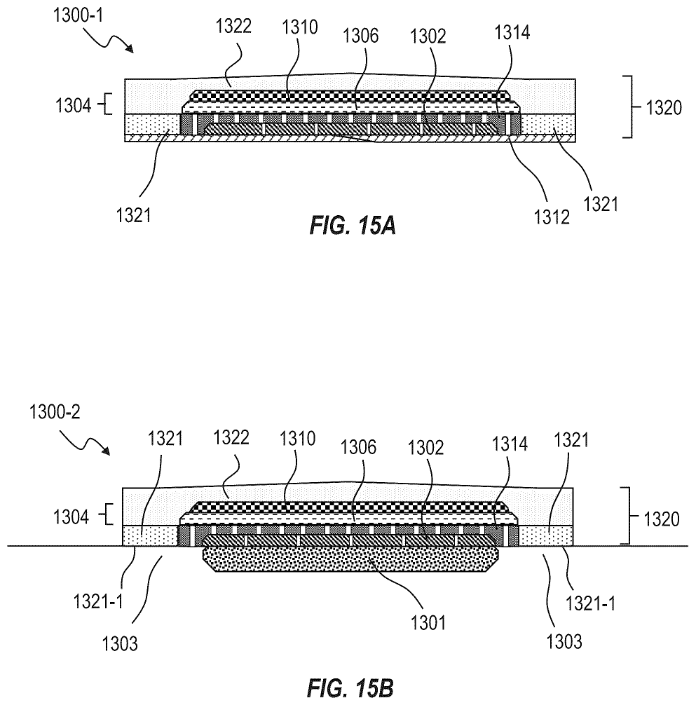

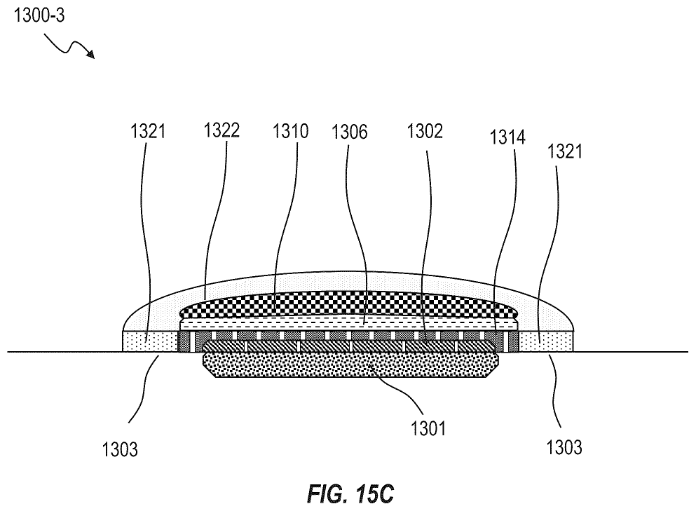

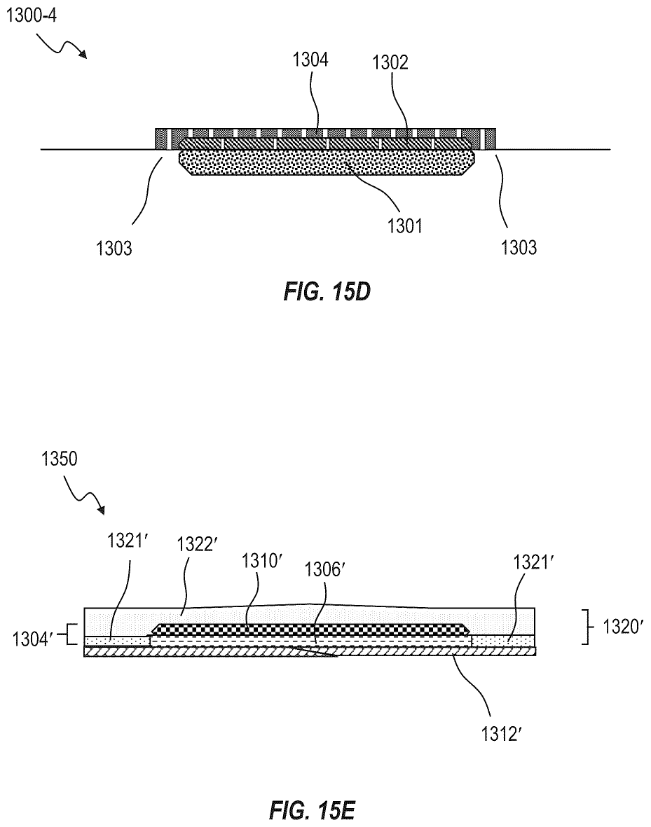

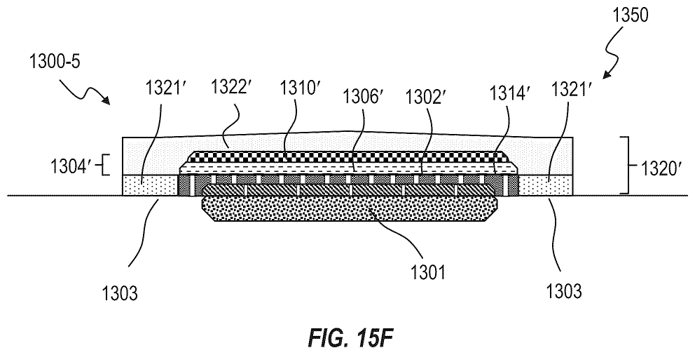

FIGS. 15A, 15B, 15C, 15D, 15E, and 15F show a progression of steps of a use of the wound treatment device 1300. In FIG. 15A, a wound treatment device 1300-1 is provided with similar features to the wound treatment device 1300 of FIGS. 13A and 13B. The release liner 1312 is intact for aseptic handling of the wound treatment device 1300 before application at the wound site. Further, the wound treatment device may be provided within a removable aseptic packaging material, which in FIG. 15A has been removed. That is, after assembling the wound treatment device, the wound treatment device may be packaged within a package of synthetic flashspun high-density polyethylene fibers (e.g., Tyvek) or other suitable packaging material.

In FIG. 15B, the release liner 1312 has been removed and the wound treatment device 1300-2 has been applied to a wound such that the skin substitute 1302 is in direct contact with the wound bed 1301. Contact surface 1321-1 of inner layer 1321 of the backing layer 1320 is contact with the skin of the subject at the peri-wound skin 1301, and as noted above, a silicone adhesive may be provided on contact surface 1321-1 to main and adhere the wound treatment device to the skin of the patient.

FIG. 15C shows the wound treatment device 1300-3 after a period of time sufficient that wound exudate has been absorbed through the contact film 1304 and both the conformable foam layer 1306 and the absorbent layer 1310 have increased in size due to absorbing exudate from the wound.

After a certain amount of time, the conformable foam layer 1306 and the absorbent layer 1310 may become sufficiently saturated, or to keep the wound treatment device 1300 fresh, the upper layers of the backing layer 1320 and the absorbent core 1304 may be removed, leaving the skin substitute 1302 and the contact film 1304 coupled to the wound bed 1301, as shown in FIG. 15D.

As shown in FIG. 15E, a replacement upper portion 1350 of a wound treatment device is provided that comprises an absorbent core 1304′, including the conformable foam layer 1306′, an absorbent layer 1310′, a backing layer 1320′ including an outer layer 1322′ and an inner layer 1321′, similar to the corresponding absorbent core 1304, conformable foam layer 1306, absorbent layer 1310, backing layer 1320, outer layer 1322, and inner layer 1321 as shown in FIGS. 13A and 13B. A release liner 1312′ is also provided in contact with the lower surface of the conformable foam layer 1306′ and the contact surfaces of the inner layer 1321′.

As shown in FIG. 15F, release liner 1312′ is removed and the wound treatment device 1300-5 is renewed by placement of the replacement upper portion 1350 to the contact film 1304 and skin substitute 1302 that had been left remaining. Thus, with the renewed wound treatment 1300-5, having a new absorbent core 1304′, additional, further exudate may be absorbed by the new absorbent core 1304′ and the upper portion that was removed can be discarded.

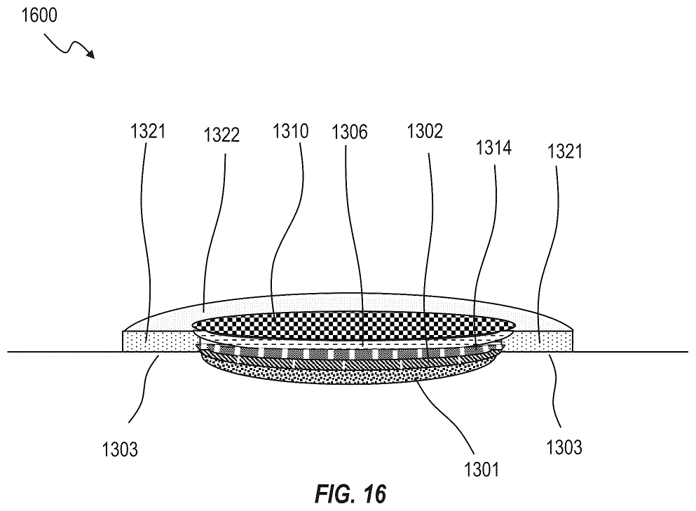

FIG. 16 illustrates a cross sectional view of the wound treatment device 1600 applied to the wound bed 1301 and peri-wound skin 1303. The skin substitute 1302 is in direct contact with the wound bed 1301, conforming to the terrain of the wound, to facilitate tissue regeneration and healing. In an embodiment, the contact film 1314, arranged above the skin substitute 1302, comprises a gently adhering silicone adhesive layer that conforms to the shape of the wound bed 101 and contacts the skin substitute 1302. The contact film 1314 facilitates the transportation of wound exudate via apertures (as discussed above) and protects the skin substitute 1302 and peri-wound skin 1303. In an embodiment, the contact film 1314 prevents absorbed wound exudate from transporting back to the wound bed 1301. The conformable foam layer 1306 works to fill the gap between the wound treatment device 1300 and wound bed 101 to reduce exudate pooling and transporting the exudate to the receptacle layer. As shown in the schematic of FIG. 16, the absorbent layer 1310 expands to entrap or lock in the absorbed exudate below the backing layer 1320.

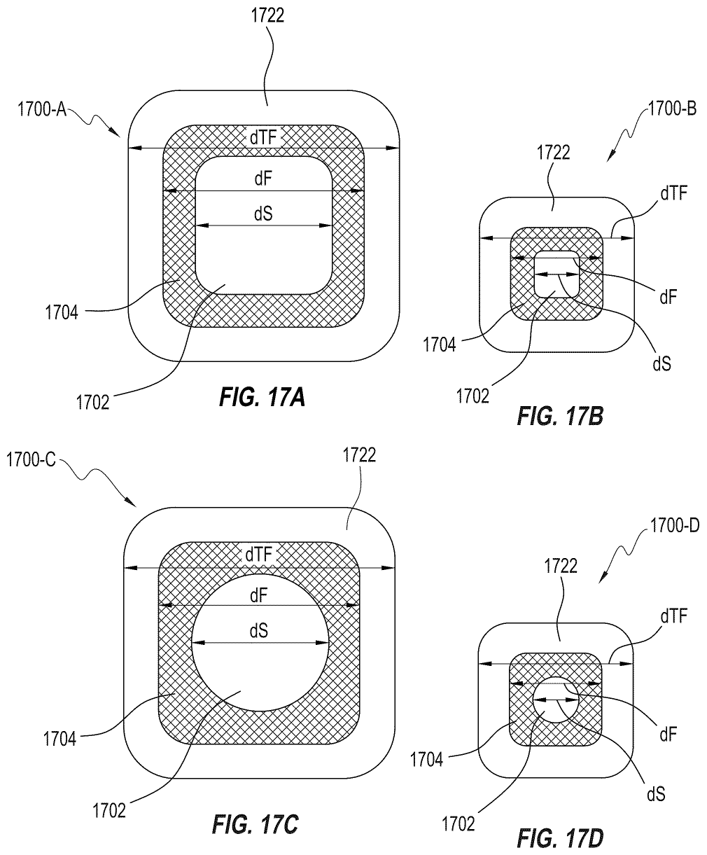

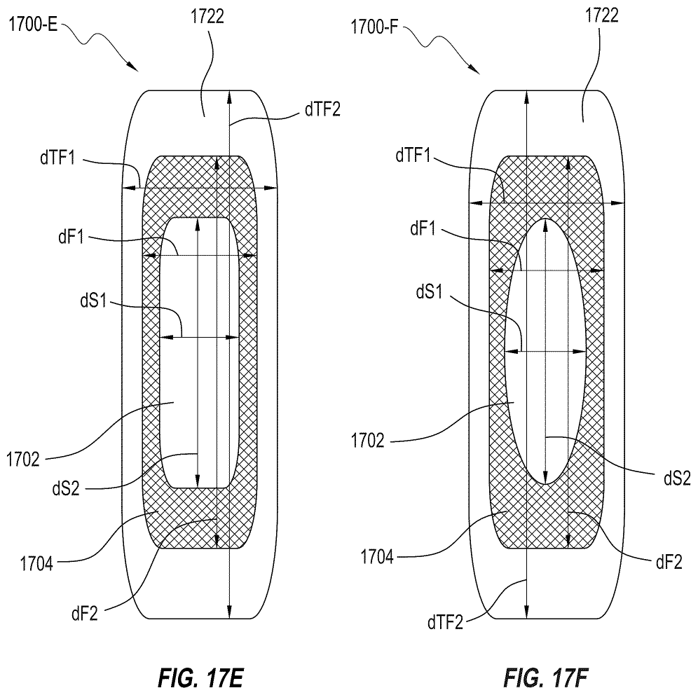

FIGS. 17A, 17B, 17C, 17D, 17E, and 17F show examples of preferable wound treatment devices 1700 provided in different geometric configurations, sizes, and shapes.

In FIGS. 17A and 17B, the wound treatment devices 1700-A and 1700-B are shown with a generally square shape, with the skin substitute 1702, absorbent core 1704, and top film 1720 each having a generally square shape and having increasing corresponding diameters dS, dF, and dTF.

In FIGS. 17C and 17D, the wound treatment devices 1700-C and 1700-D are shown with a generally square shape, with the absorbent core 1704 and top film 1720 having a generally square shape while the skin substitute 1702 have a round shape. In these examples, the skin substitutes 1702, absorbent core 1704, and top film 1720 have increasing corresponding diameters dS, dF, and dTF.

Table 1 below lists non-limiting examples of comparative sizes of the configurations shown in FIGS. 17A-17D.

TABLE 1

| | | | | | | | Fish |
|---|---|---|---|---|---|---|---|
| | | | | | | Fish | to |
| | | Border | | Foam | Fish | Skin | Foam |
| | Border | Radius | Foam | Radius | Skin | Radius | Ratio |
| 17.5 Round | 175 | 32 | 130 | 122 | 90 | | 38% |
| 17.5 Square | 175 | 32 | 130 | 22 | 90 | 17 | 48% |
| 15 Round | 150 | 28 | 105 | 18 | 70 | | 35% |
| 15 Square | 150 | 28 | 105 | 18 | 70 | 13 | 44% |
| 12.5 Round | 125 | 25 | 85 | 15 | 50 | | 27% |
| 12.5 Square | 125 | 25 | 85 | 15 | 50 | 10 | 35% |
| 10 Round | 100 | 20 | 60 | 10 | 30 | | 20% |
| 10 Square | 100 | 20 | 60 | 10 | 30 | 5 | 25% |

In the examples shown in FIGS. 17E and 17F, elongated wound treatment devices are shown, with wound treatment device 1700-E having a rectangular shape and wound treatment device 1700-F having a generally rectangular shape with a skin substitute having an elliptical shape. In each of wound treatment devices 1700-E and 1700-F, the skin substitutes 1702, absorbent core 1704, and top film 1720 have increasing corresponding minor diameters dS1, dF1, and dTF1 and increasing corresponding major diameters dS2, dF2, and dTF2. Although not shown, in other embodiments, the wound treatment device may have a non-symmetric shape.

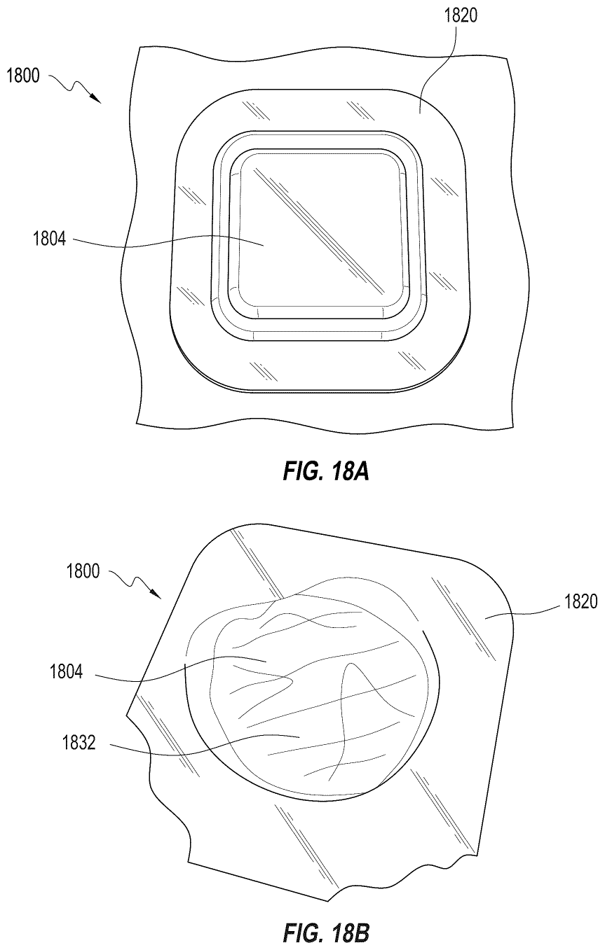

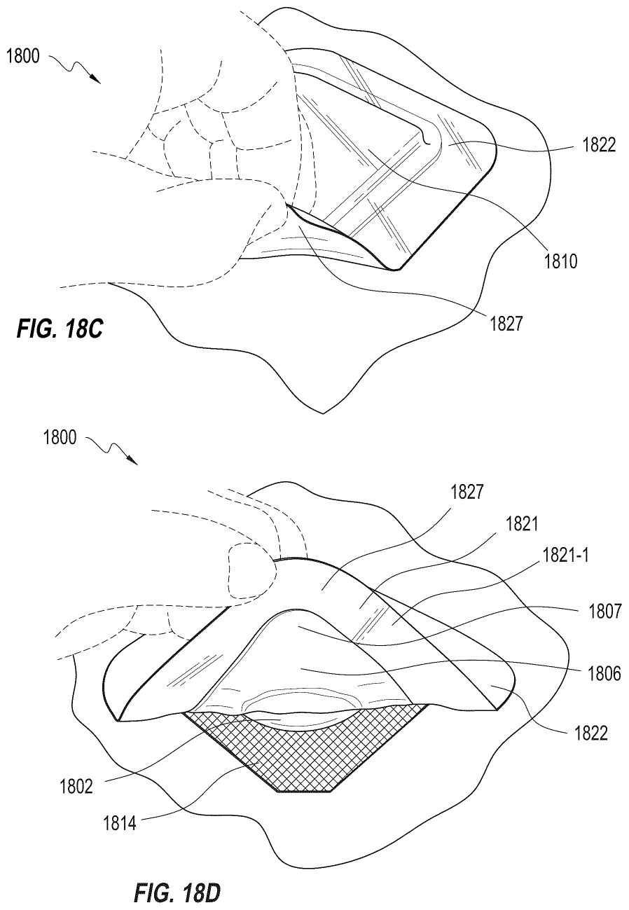

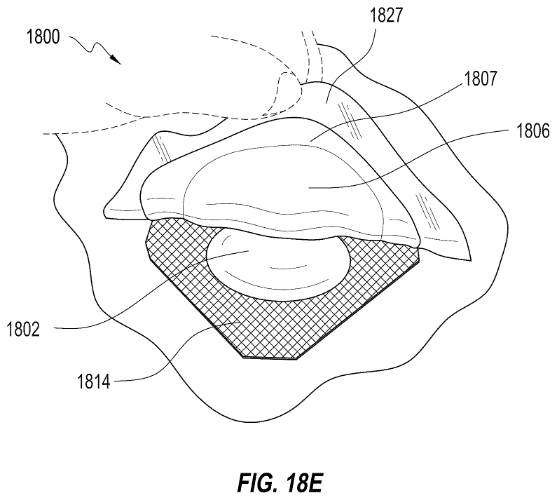

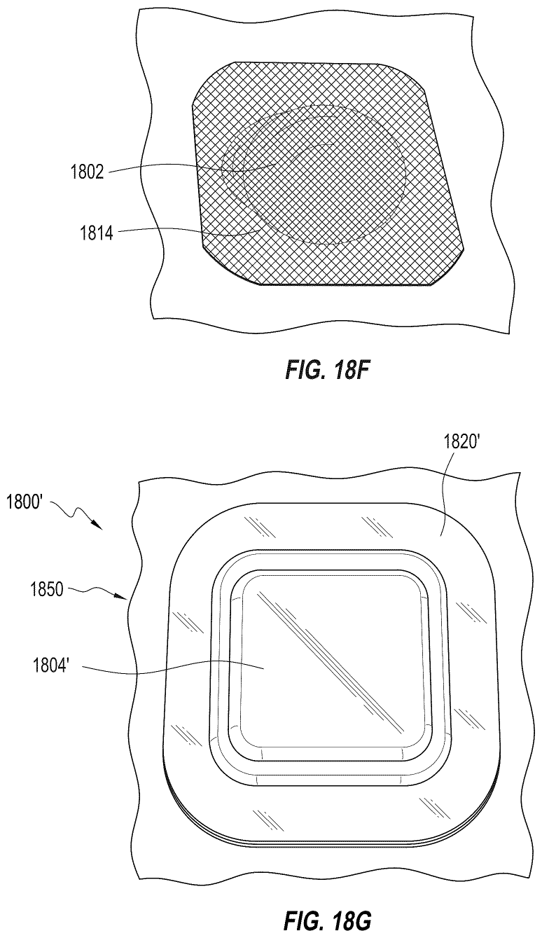

FIGS. 18A, 18B, 18C, 18D, 18E, 18F, and 18G show another progression of steps of a use of the wound treatment device 1800. In FIG. 18A, a wound treatment device 1800 has been applied over a theoretical wound. The absorbent core 1804 and top layer 1820 are identified in FIG. 18A.

In FIG. 18B, the absorbent core 1804 has absorbed a sufficient among of exudate 1832.

In FIGS. 18C, 18D, and 18E, a corner of the upper portion of the wound treatment device 1800, including a corner 1807 of the absorbent core 1804 and a corner 1827 of the top layer 1820 have been progressively lifted, separating the upper portion including the top layer 1820 and the absorbent core 1804 from the remaining contact film 1814 and skin substitute 1802 that are left in contact with the wound. The contact surface 1821-1 of the inner layer 1821 of the top layer 1820 is separated from the theoretical peri-wound skin and the inner portion of the conformable foam layer 1806 is separated form the upper surface of the contact film 1804, exposing the remaining contact film 1804 and the skin substitute 1802 arranged below the remaining contact film 1804.

In FIG. 18F, the upper potion including the top layer 1820 and the absorbent core 1804 have been entirely removed and discarded, leaving exposed the remaining contact film 1804 and the skin substitute 1802 arranged below the remaining contact film 1804.

Lastly, in FIG. 18G, a replacement upper portion 1850 of a wound treatment device has been placed over the remaining contact film 1804 and skin substitute 1802, similar to the replacement upper portion 1650 shown in in FIG. 15F, with the replacement upper portion 1850 including a new absorbent core 1804′ and a new top layer 1820′. This embodiment is particularly advantageous as it permits replacement of the absorbent core of the wound treatment device without having prematurely remove or replace the skin substitute that is in contact with the wound bed.

As noted above, the skin substitute may be or include an acellular dermal matrix which is often in a dehydrated form. For example, in a preferred embodiment, the skin substitute, or skin graft material, is an acellular dermal matrix that comprise decellularized, lyophilized fish skin. As a preferred and exemplary embodiment, the skin substitute is a lyophilized matrix product derived from intact, decellularized fish skin is described in U.S. Pat. No. 8,613,957 B2, and even more preferably from the minimally processed skin of wild-caught Atlantic cod originating from Iceland. Due to the dehydration of the skin substitute, preferably the skin substitute is rehydrated before application. However, it is also noted that a scaffold material used as a skin substitute does not necessarily require rehydration. But rehydration of a dehydrated skin substitute material has been found by the inventors to—at times—be problematic in a device wherein the skin substitute is coupled to an absorbent material, such as the absorbent core as described herein.

To address this problem, further features and embodiments of a wound treatment device or wound dressing are described herein having a hydration barrier provided between the skin substitute and the absorbent core or the hydration barrier is arranged between the skin substitute and at least a portion of the absorbent core. The hydration barrier delays passage of moisture, liquid, and/or vapor from a hydration of the skin substitute to the absorbent core or the at least portion of the absorbent core. Preferably the hydration barrier is a water-dissolvable, water-soluble, water-miscible, and/or water-susceptible barrier that does not immediately dissolve upon exposure to water but that dissolves or breaks down upon continued exposure to water or other chemicals within the wound over time. Although the hydration barrier may be dissolvable by water, other compositions of a hydration barrier may be used that would be broken down, dissolved, removed, or changed upon exposure to chemicals within the wound during healing of the wound but after hydration of the skin substitute that is coupled to the absorbent core. In another embodiment, the hydration barrier is not dissolvable, but remains intact, or partially intact, or substantially intact that is configured to delay passage of moisture, liquid, and/or vapor from a hydration of the skin substitute to the absorbent core or the at least portion of the absorbent core. The hydration barrier may be a layer or multi-layer structure, such as a polymer layer or polymer multi-layer structure. In another embodiment, the hydration barrier is a removable or alterable layer or multi-layer structure or device, such as an array of micro- or nano-valves or porous structure that delays or prevents passage of moisture, liquid, and/or vapor from a hydration of the skin substitute to the absorbent core or the at least portion of the absorbent core. The hydration barrier may be configured to be removed or altered after hydration of the skin substitute such that before and during hydration the barrier layer delays or prevents passage of moisture, liquid, and/or vapor from a hydration of the skin substitute to the absorbent core or the at least portion of the absorbent core, but upon removal or alteration, such as manual removal by a practitioner, moisture, liquid, and/or vapor is permitted to pass to the absorbent core.

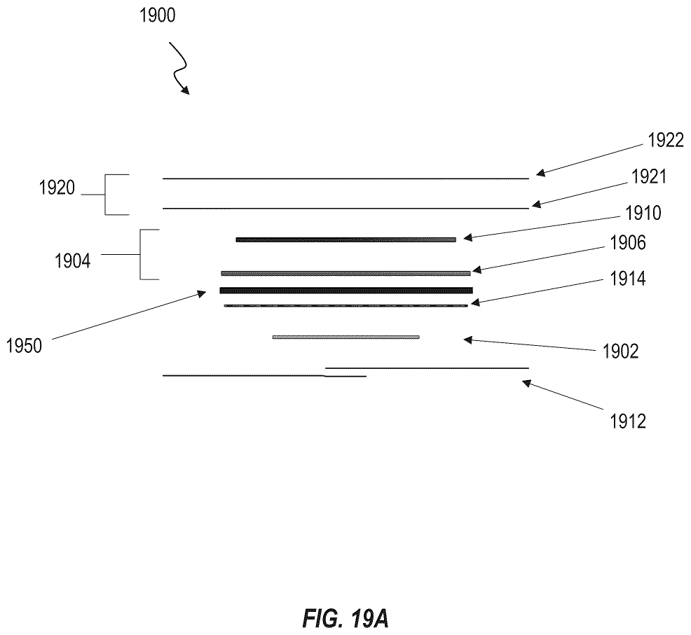

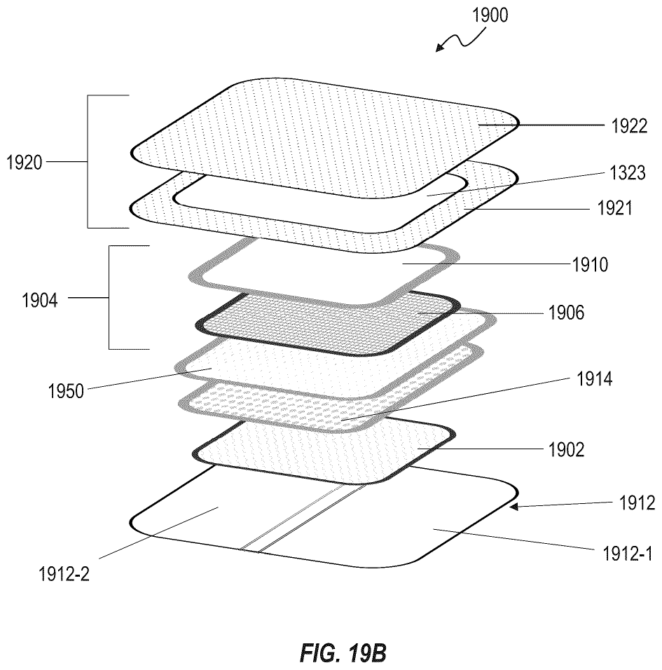

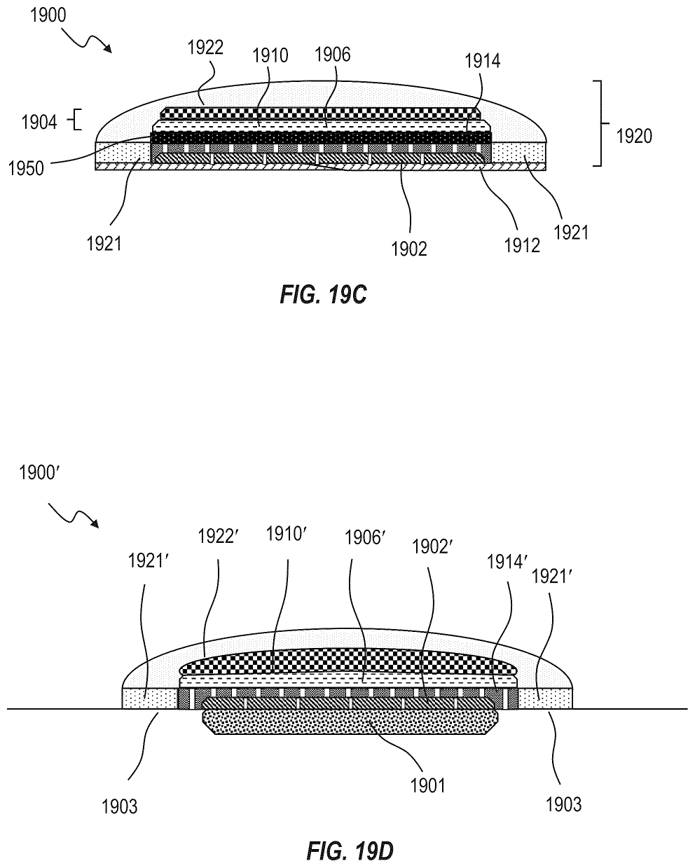

FIGS. 19A, 19B, and 19C each depict a schematic of a wound treatment device 1900 according to another embodiment that includes a non-limiting example of a hydration barrier. In the embodiment of FIGS. 19A and 19B, the wound treatment device 1900 is a wound dressing comprising an intact skin substitute 1902 used to support tissue regeneration, and particularly skin regeneration, in a wound.

The wound treatment device 1900 comprises a skin substitute 1902 and an absorbent core 1904. A hydration barrier 1950 is provided between the skin substitute 1902 and the absorbent core 1904. The skin substitute 1902 adheres to a contact layer or contact film 1914 that is also disposed between the skin substitute 1902 and absorbent core 1904, such that the hydration barrier 1950 is arranged between the contact film 1914 and the absorbent core 1904. The absorbent core 1904 comprises a conformable foam layer 1906 and an absorbent layer 1910 for absorbing and containing wound exudate. The wound treatment device 1900 includes a backing layer 1920 as a backing layer or top layer or top film that is provided to protect the lower layers (1902, 1914, 1904) from damage and contamination and also to protect the wound bed and peri-wound skin. The backing layer 1920 is preferably a multi-layer film including, in a preferred embodiment, an outer layer 1922 that overlies an inner layer 1921. The inner layer 1921 defines an inner, unoccupied space 1923 such that the backing layer 1920 is provided with a concavity at space 1923. In other words, the inner layer 1921 has a central portion removed at space 1921 such that only the perimeter portion remains. The wound treatment device 1900 also comprises a release liner 1912 for aseptic handling of the wound treatment device 1900 before application at the wound site. The release liner 1912 preferably includes two portions 1912-1, 1912-2, which meet and make contact with each other.

As shown in FIG. 19D, the hydration barrier 1950 remains intact or substantially intact during rehydration of the skin substitute 1902, such that upon removal of the release liner, and hydration, for example, by dousing, misting, squirting, or other forms of hydration of the skin substitute the water used to hydrate the skin substitute does not contact the materials of the absorbent core 1904, or at least water used in the hydration of the skin substitute 1902 does not readily or substantially make contact the materials of the absorbent core 1904 due to the presence of the hydration barrier 1950. However, after the wound treatment device 1900′ has been in place on a treated wound 1901, the hydration barrier 1950 is dissolved, broken down, or otherwise removed, or substantially removed, such that the absorbent core 1904′, and particularly the conformable layer 1906′ comes into contact with the contact film 1914′, and due to the provided tackiness of the contact film 1914′, and particularly due to the tackiness of the top surface of the top layer of the contact film 1914′, upon dissolving of the water barrier 1950, lasting and effective contact between the contact film 1914′ and the conformable layer 1906′ is maintained.

As shown in FIG. 19D, the hydration barrier 1950 remains intact or substantially intact during rehydration of the skin substitute 1902, such that upon removal of the release liner, and hydration, for example, by dousing, misting, squirting, or other forms of hydration of the skin substitute the water used to hydrate the skin substitute does not contact the materials of the absorbent core 1904, or at least water used in the hydration of the skin substitute 1902 does not readily or substantially make contact the materials of the absorbent core 1904 due to the presence of the hydration barrier 1950. However, after the wound treatment device 1900′ has been in place on a treated wound 1901, the hydration barrier 1950 is dissolved, broken down, or otherwise removed, or substantially removed, such that the absorbent core 1904′, and particularly the conformable layer 1906′ comes into contact with the contact film 1914′, and due to the provided tackiness of the contact film 1914′, and particularly due to the tackiness of the top surface of the top layer of the contact film 1914′, upon dissolving of the water barrier 1950, lasting and effective contact between the contact film 1914′ and the conformable layer 1906′ is maintained.

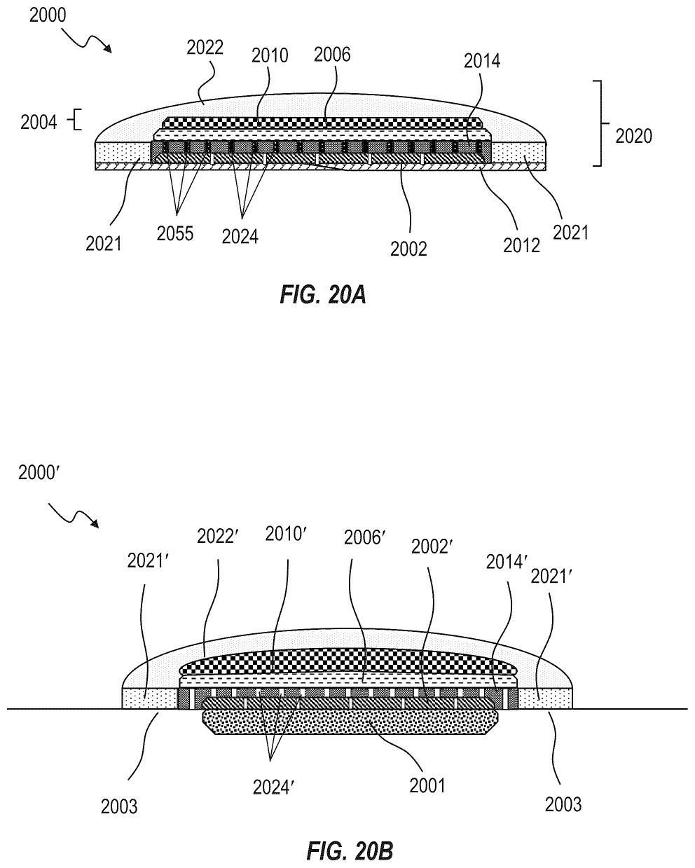

FIG. 20 depicts a schematic of a wound treatment device 2000 according to another embodiment. In the embodiment of FIG. 20A, the wound treatment device 2000 is a wound dressing comprising an intact skin substitute 2002 used to support tissue regeneration, and particularly skin regeneration, in a wound.

The wound treatment device 2000 comprises a skin substitute 2002 and an absorbent core 2004 coupled to the skin substitute. A contact layer or contact film 2014 is provided between the skin substitute 2002 and the absorbent core 2004 such that the skin substitute 2002 and the absorbent core 2004 are in contact with opposing surfaces of the contact film 2014. The absorbent core 2004 comprises a conformable foam layer 2006 and an absorbent layer 2010 for absorbing and containing wound exudate. The wound treatment device 2000 includes a backing layer 2020 as a backing layer or top layer or top film that is provided to protect the lower layers (2002, 2014, 2004) from damage and contamination and also to protect the wound bed and peri-wound skin. The backing layer 2020 is preferably a multi-layer film including, in a preferred embodiment, an outer layer 2022 that overlies an inner layer 2021. The inner layer 2021 defines an inner, unoccupied space 2023 such that the backing layer 2020 is provided with a concavity. In other words, the inner layer 2021 has a central portion removed at space 2021 such that only the perimeter portion remains. The wound treatment device 2000 also comprises a release liner 2012 for aseptic handling of the wound treatment device 2000 before application at the wound site. The release liner 2012 preferably includes two portions 2012-1, 2012-2, which meet and make contact with each other.

As shown in FIG. 20A, the contact film 2014 is provided with apertures 2024 configured to allow passage of wound exudate through the contact film 2014 to be absorbed by the absorbent core 2004, similar to and sharing characteristics of the apertures through the contact layer or contact film as described herein. In this embodiment of FIG. 20A, the apertures 2024 are filled with a barrier material 2055 that, similar to the material of the hydration barrier 1950 in the embodiments of FIGS. 19A to 19C, is filled completely, substantially or at least sufficiently to prevent penetration by water through the apertures 2024 to be absorbed by the absorbent core 2004. After rehydrating the wound treatment device 2000, the wound treatment device 2000′ is placed on the wound 2001. Upon exposure to the environment of the wound, the material 2055 of the hydration barrier 2055 is dissolved, broken down, or otherwise removed, or substantially removed, such that exudate from the wound is permitted to pass through the apertures 2024′ to be absorbed by the absorbent core 2004′, and particularly through the conformable layer 2010′ and absorbed by the receptacle layer 2010′ of the absorbent core 2004′.

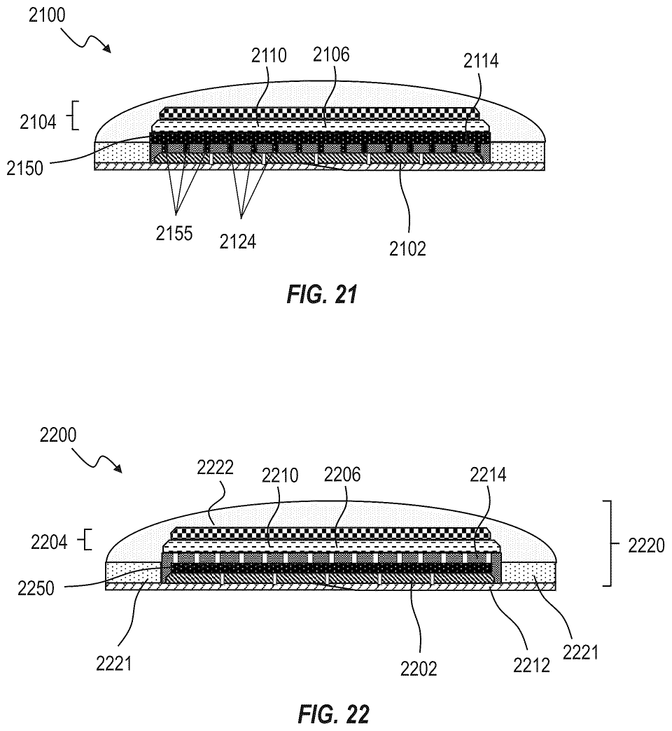

In another embodiment, as shown in FIG. 21, the wound treatment device includes both a barrier layer 2150 provided between the skin substitute 2102 and the absorbent core 2104, and particularly the barrier layer 2150 is provided between the contact film 2114 and the conformable layer 2106 of the absorbent core 2104. Additionally, barrier material 2155, of a similar or different composition as the barrier layer 2150, but similarly dissolvable or otherwise removable during exposure to wound exudate with time, is provided in apertures 2124 of the contact film 2114. The combination of the barrier layer 2150 and the barrier material 2155 prevents absorption or water, or substantially prevents absorption of water during rehydration of the skin substitute 2102. However, the dissolvability or susceptibility of the material of the barrier layer 2150 and the barrier material 2155 permits eventual absorption of the wound exudate by the absorbent core 2104.

FIG. 22 shows a schematic of a wound treatment device 2200 according to another embodiment. In the embodiment of FIG. FIG. 22, the wound treatment device 2200 is a wound dressing comprising an intact skin substitute 2202 used to support tissue regeneration, and particularly skin regeneration, in a wound.

The wound treatment device 2200 differs from that of the embodiments of 19A to 19C in that the barrier layer 2250 is provided between the skin substitute 2202 and the contact film 2214. That is, the wound treatment device 2200 comprises a skin substitute 2202 and an absorbent core 2204. The hydration barrier 2250 is provided between the skin substitute 1902 and the contact film 2014. The absorbent core 2204 comprises a conformable foam layer 2206 and an absorbent layer 2210 for absorbing and containing wound exudate. The wound treatment device 2200 includes a backing layer 2220 as a backing layer or top layer or top film that is provided to protect the lower layers (2202, 2214, 2204) from damage and contamination and also to protect the wound bed and peri-wound skin. The backing layer 2220 is preferably a multi-layer film including, in a preferred embodiment, an outer layer 2222 that overlies an inner layer 2221. Similar to other embodiments, the wound treatment device 2200 also comprises a release liner 2212 for aseptic handling of the wound treatment device 1900 before application at the wound site.

The hydration barrier 2250 remains intact or substantially intact during rehydration of the skin substitute 2202, such that upon removal of the release liner, and hydration, for example, by dousing, misting, squirting, or other forms of hydration of the skin substitute the water used to hydrate the skin substitute does not contact the materials of the absorbent core 2204, or at least water used in the hydration of the skin substitute 2202 does not readily or substantially make contact the materials of the absorbent core 2204 due to the presence of the hydration barrier 2250. However, after the wound treatment device 2200 has been in placed on a treated wound, the hydration barrier 2250 is dissolved, broken down, or otherwise removed, or substantially removed, such that the skin substitute 2202 comes into contact with the contact film 2214, and due to the provided tackiness of the contact film 2214, and particularly due to the tackiness of the top bottom surface of the bottom layer of the contact film 2214, upon dissolving of the water barrier 2250, lasting and effective contact between the contact film 2214 and the skin substitute 2202 is made, particularly due to a force applied by the conformable layer 2206.

The hydration barrier 2250 remains intact or substantially intact during rehydration of the skin substitute 2202, such that upon removal of the release liner, and hydration, for example, by dousing, misting, squirting, or other forms of hydration of the skin substitute the water used to hydrate the skin substitute does not contact the materials of the absorbent core 2204, or at least water used in the hydration of the skin substitute 2202 does not readily or substantially make contact the materials of the absorbent core 2204 due to the presence of the hydration barrier 2250. However, after the wound treatment device 2200 has been in place on a treated wound, the hydration barrier 2250 is dissolved, broken down, or otherwise removed, or substantially removed. and exudate from the wound may pass through apertures of the contact film 2214 and absorbed by the absorbent core 2204.

As described herein, the barrier layer and barrier material may be made from or composed of a variety of materials. In a preferred embodiment, the material of the barrier layer or barrier material within the apertures of the contact film are water-dissolvable, water soluble, or water susceptible. The material of the barrier layer and barrier material placed within the apertures of the contact film is a sacrificial moisture barrier as a coating or “mask” between the foam layers of the absorbent core. The material of the barrier layer and the barrier material is biocompatible with the wound or are not detrimental to healing of the wound. Such materials may include, but are not limited to gelatine, piscine gelatin, denatured collagen, biocompatible polymeric materials, starches, collagen, water-soluble carrageenan, pectin, xanthan gum, guar gum, or other gums, or combinations thereof. The significance of the material of the hydration barrier is that the material is not so readily dissolvable by water that the material would dissolve or otherwise break down or substantially break down during hydration of the skin substitute in a way that would permit contact or substantial contact between the water used in the hydration of the skin substitute and the absorbent core. The thickness, density and extent of the barrier layer is influenced or affected by its persistence and thus control the dwell time between hydration of the skin substitute and access by water to the foam of the absorbent core. Alternatively, and preferably, the hydration barrier may be confined to the pores or apertures of the polyurethane of the contact layer so as to permit contact between the silicone adhesive layer and the foam and skin substitute respectively and not alter adhesion between the layers of the composite upon hydration. In other embodiments, the hydration barrier may encapsulate a substantial portion of the product to further protect the foam layers of the absorbent core from exposure to moisture during hydration of the skin substitute.

Further, other materials may be contemplated, that, although not water soluble or water susceptible, may be broken down, or removed, or substantially broken down upon exposure to a chemical, solvent, or enzyme within the wound environment or wound exudate.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

### Combinability of Embodiments and Features

This disclosure provides various examples, embodiments, and features which directed to wound treatment devices, kits, systems, and methods. Unless expressly stated, or unless such examples, embodiments, and features would be mutually exclusive, the various examples, embodiments, and features disclosed herein should be understood to be combinable with other examples, embodiments, or features described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

It is to be understood that not necessarily all objects or advantages may be achieved under an embodiment of the disclosure. Those skilled in the art will recognize that the wound treatment devices and methods for making the same may be embodied or carried out in a manner that achieves or optimizes one advantage or group of advantages as taught herein without achieving other objects or advantages as taught or suggested herein.

### Combinability of Embodiments and Features

This disclosure provides various examples, embodiments, systems, devices, and methods for wound treatment. In addition to the above disclosure, further embodiments and examples include the following groups and enumerated embodiments.

### Group 1—a Wound Treatment Device or, in Other Words, a Wound Dressing, and Corresponding Kit and Method

1-1. A wound treatment device comprising: a skin substitute configured to be placed in contact with a wound; a contact film arranged in contact with the skin substitute between the absorbent core and the skin substitute; and a replaceable portion arranged overlaying the contact film, the replaceable portion including an absorbent core configured to absorb exudate from the wound and a top film overlying the absorbent core, such that the absorbent core is arranged between the contact film and the top film, wherein the replaceable portion is separable from the contact film such that the replaceable portion may be removed from the contact film without disrupting or removing the skin substitute.

1-2. The wound treatment device according to any one or a combination of one or more of 1-1 above and/or 1-3 to 1-31 below, wherein the absorbent core comprises a conformable foam layer to conform to a shape of a wound bed.

1-3. The wound treatment device according to any one or a combination of one or more of 1-1 to 1-2 above and/or 1-4 to 1-31 below, wherein the skin substitute includes an acellular dermal matrix that comprises decellularized fish skin.

1-4. The wound treatment device according to any one or a combination of one or more of 1-1 to 1-3 above and/or 1-5 to 1-31 below, wherein the skin substitute includes decellularized fish skin.

1-5. The wound treatment device according to any one or a combination of one or more of 1-1 to 1-4 above and/or 1-6 to 1-31 below, wherein the skin substitute comprises lipids from a lipid layer of the decellularized fish skin.

1-6. The wound treatment device according to any one or a combination of one or more of 1-1 to 1-5 above and/or 1-7 to 1-31 below, wherein the skin substitute comprises an extracellular matrix product in a three-dimensional form of particles, or a sheet, or a mesh.

1-7. The wound treatment device according to any one or a combination of one or more of 1-1 to 1-6 above and/or 1-8 to 1-31 below, wherein the contact film is a multi-layer film including a core layer, and first outer layer provided on a first surface of the core layer and a second outer layer provided on a second surface of the core layer, the first surface of the core layer being opposite from the second surface of the core layer, the first outer layer is in contact with and removable adhered to the skin substitute and the second outer layer is in contact with the absorbent core.

1-8. The wound treatment device according to any one or a combination of one or more of 1-1 to 1-7 above and/or 1-9 to 1-31 below, wherein the first outer layer is a silicone adhesive layer.

1-9. The wound treatment device according to any one or a combination of one or more of 1-1 to 1-8 above and/or 1-10 to 1-31 below, wherein the second outer layer is another silicone adhesive layer.

1-10. The wound treatment device according to any one or a combination of one or more of 1-1 to 1-9 above and/or 1-11 to 1-31 below, wherein the core layer of the contact film is made of polyethylene terephthalate (PET).

1-11. The wound treatment device according to any one or a combination of one or more of 1-1 to 1-10 above and/or 1-12 to 1-31 below, wherein the absorbent core further comprises a absorbent layer.

1-12. The wound treatment device according to any one or a combination of one or more of 1-1 to 1-11 above and/or 1-13 to 1-31 below, wherein the contact film has a predetermined porosity.

1-13. The wound treatment device according to any one or a combination of one or more of 1-1 to 1-13 above and/or 1-14 to 1-31 below, wherein the predetermined porosity defines a distribution of apertures over a surface area of the contact film.

1-14. The wound treatment device according to any one or a combination of one or more of 1-1 to 1-13 above and/or 1-15 to 1-31 below, wherein the skin substitute is fenestrated.

1-15. The wound treatment device according to any one or a combination of one or more of 1-1 to 1-14 above and/or 1-16 to 1-31 below, wherein the absorbent layer comprises a superabsorbent polymer.

1-16. The wound treatment device according to any one or a combination of one or more of 1-1 to 1-15 above and/or 1-17 to 1-31 below, wherein the absorbent core comprises microcapillaries adapted to promote vertical absorption of wound exudate.

1-17. The wound treatment device according to any one or a combination of one or more of 1-1 to 1-16 above and/or 1-18 to 1-31 below, wherein the top film includes an outer layer and an inner layer, the outer layer overlaying the inner layer, wherein the inner layer defines a central space through which the an inner surface of the outer layer contacts the absorbent core.

1-18. The wound treatment device according to any one or a combination of one or more of 1-1 to 1-17 above and/or 1-19 to 1-31 below, wherein a contact surface of the inner layer is configured to contact skin around the perimeter of the wound bed.

1-19. The wound treatment device according to any one or a combination of one or more of 1-1 to 1-18 above and/or 1-20 to 1-31 below, wherein the outer layer of the top film is adhered to the absorbing layer of the absorbent core.

1-20. The wound treatment device according to any one or a combination of one or more of 1-1 to 1-19 above and/or 1-21 to 1-31 below, wherein a silicone adhesive is provided on the contact surface of the inner layer of the top film.

1-21. The wound treatment device according to any one or a combination of one or more of 1-1 to 1-20 above and/or 1-22 to 1-31 below, wherein a silicone adhesive is provided on the contact surface of the inner layer of the top film, the silicone adhesive being configured to removably adhere the inner layer to the skin around the perimeter of the wound bed.

1-22. The wound treatment device according to any one or a combination of one or more of 1-1 to 1-21 above and/or 1-23 to 1-31 below, wherein the top film is liquid impermeable and vapor permeable.

1-23. The wound treatment device according to any one or a combination of one or more of 1-1 to 1-22 above and/or 1-24 to 1-31 below, wherein the top film is air-tight and is adapted to create a seal about the wound bed.

1-24. The wound treatment device according to 1, further comprising a barrier layer arranged between the skin substitute and the absorbent core.

1-25. The wound treatment device according to any one or a combination of 24 above and/or 26-31 below, wherein the barrier layer is arranged between the contact film and the absorbent core.

1-26. The wound treatment device according to 1-24 to 1-25 above or 1-27 to 1-31 below, wherein the barrier layer is arranged between the contact film and the skin substitute.

1-27. The wound treatment device according to 1-24 to 1-26 above or 1-28 to 1-31 below, wherein the barrier layer includes a barrier material provided within aperture that extend through the contact film.

1-28. The wound treatment device according to 1-24 to 1-27 above or 1-29 to 1-31 below, wherein the barrier layer includes a material that is a water-soluble, water-soluble, water-miscible, and/or water-susceptible.

1-29. The wound treatment device according to 1-24 to 1-28 above or 1-30 to 1-31 below, wherein the barrier layer includes gelatin.

1-30. The wound treatment device according to 1-24, wherein the barrier layer includes a material that is susceptible to be broken down upon exposure to wound exudate or the wound environment.

1-31. The wound treatment device according to 30, wherein the barrier layer includes a material that non-water soluble material.

1-32. A negative pressure wound therapy apparatus comprising the wound treatment device according to any one or a combination of one or more of 1-1 to 1-31 above.

1-33. A pre-packaged kit comprising the wound treatment device according to any one or a combination of one or more of 1-1 to 1-31 above, wherein the wound treatment device is provided within an aseptic packaging material.

1-34. A method of treating a wound by applying the wound treatment device according to any one or a combination of one or more of 1-1 to 1-31 above over a wound bed.

1-35. The method of treating a wound according to 1-34 above, further comprising removing a replaceable portion of the wound treatment device, the replaceable portion including the absorbent core and the top film after a period of time, and leaving the remaining portion intact at the wound bed, the remaining portion including the contact film and the skin substitute.

1-36. The method according to 35 above, further comprising applying a second replaceable portion over the remaining portion, the second replaceable portion comprising a second absorbent core configured to absorb further exudate from the wound and a second top film overlying the second absorbent core, such that the second absorbent core is arranged between the contact film and the second top film.

### Group 2—Another Wound Treatment Device or, in Other Words, a Wound Dressing, and Corresponding Kit and Method

2-1. A wound treatment device comprising: a skin substitute; an absorbent core coupled to the skin substitute; wherein the absorbent core comprises a conformable foam layer to conform to a shape of a wound bed.

2-2. The wound treatment device according to any one or a combination of one or more of 2-1 above and/or 2-3 to 2-22 below, wherein the skin substitute includes an acellular dermal matrix that comprises decellularized fish skin.

2-3. The wound treatment device according to any one or a combination of one or more of 2-1 to 2-2 above and/or 2-4 to 2-22 below, wherein the skin substitute includes decellularized fish skin.

2-4. The wound treatment device according to any one or a combination of one or more of 2-1 to 2-3 above and/or 2-5 to 2-22 below, wherein the skin substitute comprises lipids from a lipid layer of the decellularized fish skin.

2-5. The wound treatment device according to any one or a combination of one or more of 2-1 to 2-4 above and/or 2-6 to 2-22 below, wherein the skin substitute comprises an extracellular matrix product in a three-dimensional form of particles, or a sheet, or a mesh.

2-6. The wound treatment device according to any one or a combination of one or more of 2-1 to 2-5 above and/or 2-7 to 2-22 below, further comprising a polymer contact layer, wherein the polymer contact layer is integrally bonded to the absorbent core and removably adhered to the skin substitute.

2-7. The wound treatment device according to any one or a combination of one or more of 2-1 to 2-6 above and/or 2-8 to 2-22 below, wherein the absorbent core further comprises a receptacle layer.

2-8. The wound treatment device according to any one or a combination of one or more of 2-1 to 2-7 above and/or 2-9 to 2-22 below, wherein the polymer contact layer has a predetermined porosity.

2-9. The wound treatment device according to any one or a combination of one or more of 2-1 to 2-8 above and/or 2-10 to 2-22 below, wherein the predetermined porosity defines a distribution of apertures over a surface area of the polymer contact layer.

2-10. The wound treatment device according to any one or a combination of one or more of 2-1 to 2-9 above and/or 2-11 to 2-22 below, wherein the receptacle layer comprises a superabsorbent polymer.

2-11. The wound treatment device according to any one or a combination of one or more of 2-1 to 2-10 above and/or 2-12 to 2-22 below, wherein the absorbent core comprises microcapillaries adapted to promote vertical absorption of wound exudate.

2-12. The wound treatment device according to any one or a combination of one or more of 2-1 to 2-11 above and/or 2-13 to 2-22 below, wherein each microcapillary comprises a vertical channel and lateral pockets.

2-13. The wound treatment device according to any one or a combination of one or more of 2-1 to 2-12 above and/or 2-14 to 2-22 below, wherein the absorbent core comprises at least one cavity for receiving at least one skin substitute portion to mechanically couple the skin substitute to the absorbent core.

2-14. The wound treatment device according to any one or a combination of one or more of 2-1 to 2-13 above and/or 2-15 to 2-22 below, further comprises a suture to mechanically couple the skin substitute to the absorbent core.

2-15. The wound treatment device according to any one or a combination of one or more of 2-1 to 2-14 above and/or 2-16 to 2-22 below, wherein the suture is biodegradable and adapted to dissolve over a period of time to allow for separation of the skin substitute from the absorbent core.

2-16. The wound treatment device according to 2-1, further comprising a barrier layer arranged between the skin substitute and the absorbent core.

2-17. The wound treatment device according to any one or a combination of 2-16 above and/or 2-18 to 2-22 below, wherein the barrier layer is arranged between the contact film and the absorbent core.

2-18. The wound treatment device according to any one or a combination of 2-16 to 2-17 above and/or 2-19 to 2-22 below, wherein the barrier layer is arranged between the contact film and the skin substitute.

2-19. The wound treatment device according to any one or a combination of 2-16 to 2-18 above and/or 2-20 to 2-22 below, wherein the barrier layer includes a barrier material provided within aperture that extend through the contact film.

2-20. The wound treatment device according to any one or a combination of 2-16 to 2-19 above and/or 2-21 to 2-22 below, wherein the barrier layer includes a material that is a water-soluble, water-soluble, water-miscible, and/or water-susceptible.

2-21. The wound treatment device according to any one or a combination of 2-16 to 2-20 above and/or 2-22 below, wherein the barrier layer includes gelatin.

2-22. The wound treatment device according to 2-21, wherein the barrier layer includes a material that is susceptible to be broken down upon exposure to wound exudate or the wound environment.

2-23. A negative pressure wound therapy apparatus comprising the wound treatment device according to any one or a combination or one or more of 2-1 to 2-22 above.

2-24. The wound treatment device of according to any one or a combination or one or more of 2-1 to 2-22 above, wherein the wound treatment device comprises a backing layer.

2-25. The wound treatment device according to any one or a combination or one or more of 2-1 to 2-22 above, wherein the backing layer is liquid impermeable and vapor permeable.

2-26. The wound treatment device according to any one or a combination or one or more of 2-1 to 2-22 above, wherein the backing layer is air-tight and is adapted to create a seal about the wound bed.

2-27. A pre-packaged kit comprising the wound treatment device according to any one or a combination or one or more of 2-1 to 2-22 above, wherein the wound treatment device is provided within a aseptic packaging material.

2-28. A method of treating a wound by applying the wound treatment device according to any one or a combination or one or more of 2-1 to 2-22 above over a wound bed.

The skilled artisan will recognize the interchangeability of some of the various disclosed features. Besides the variations described herein, other known equivalents for each feature can be mixed and matched by one of ordinary skill in this art to construct an wound treatment device and utilize a method for making the same under principles of the present disclosure.

Although this disclosure describes certain exemplary embodiments and examples of a wound treatment devices, it will be understood by those skilled in the art that the present disclosure extends beyond the specifically disclosed wound treatment device embodiments to other alternative embodiments and/or uses of the disclosure and obvious modifications and equivalents thereof. It is intended that the present disclosure should not be limited by the disclosed embodiments described above and may be extended to other applications that may employ the features described herein. It will be understood that the above described embodiments of the invention are illustrative in nature, and that modifications thereof may occur to those skilled in the art. Accordingly, this invention is not to be regarded as limited to the embodiments disclosed herein, but is to be limited only as defined in the appended claims.

1. A wound treatment device comprising:
a skin substitute;
an absorbent core, the skin substitute being coupled to the absorbent core,
wherein the absorbent core comprises a conformable foam layer to conform to a shape of a wound bed.

2. The wound treatment device according to claim 1, wherein the skin substitute includes decellularized fish skin.

3. The wound treatment device according to claim 1, wherein the skin substitute comprises an extracellular matrix product in a three-dimensional form of particles, or a sheet, or a mesh.

4. The wound treatment device according to claim 1, further comprising a polymer contact layer, wherein the polymer contact layer is integrally bonded to the absorbent core and removably adhered to the skin substitute.

5. The wound treatment device according to claim 1, wherein the absorbent core further comprises a receptacle layer.

6. The wound treatment device according to claim 1, wherein the polymer contact layer has a predetermined porosity.

7. The wound treatment device according to claim 6, wherein the predetermined porosity defines a distribution of apertures over a surface area of the polymer contact layer.

8. The wound treatment device according to claim 1, wherein the receptacle layer comprises a superabsorbent polymer.

9. The wound treatment device according to claim 8, wherein the absorbent core comprises microcapillaries adapted to promote vertical absorption of wound exudate.

10. The wound treatment device according to claim 1, wherein the wound treatment device comprises a backing layer arranged such that the absorbent core is arranged between the contact film and the backing layer, wherein the backing layer is liquid impermeable and vapor permeable, wherein the backing layer is adapted to create a seal about the wound bed.

11. The wound treatment device according to claim 1, wherein the wound treatment device comprises a hydration barrier between the skin substitute and the absorbent core or between the skin substitute and at least a portion of the absorbent core, wherein the hydration barrier delays passage of moisture, liquid, or vapor from a hydration of the skin substitute to the absorbent core or the at least portion of the absorbent core.

12. A negative pressure wound therapy apparatus comprising the wound treatment device according to claim 1.

13. A pre-packaged kit comprising the wound treatment device according to claim 1, wherein the wound treatment device is provided within an aseptic packaging material.

14. A method of treating a wound, the method comprising providing the wound treatment device according to claim 1, and
applying the wound treatment device over a wound bed.

15. A wound treatment device comprising:
a skin substitute configured to be placed in contact with a wound;
a contact film arranged in contact with the skin substitute between the absorbent core and the skin substitute; and
a replaceable portion arranged overlaying the contact film, the replaceable portion including an absorbent core configured to absorb exudate from the wound and a top film overlying the absorbent core, such that the absorbent core is arranged between the contact film and the top film,
wherein the replaceable portion is separable from the contact film such that the replaceable portion may be removed from the contact film without disrupting or removing the skin substitute.

16. The wound treatment device according to claim 15, wherein the absorbent core comprises a conformable foam layer configured to conform to a shape of a wound bed.

17. The wound treatment device according to claim 15, wherein the skin substitute includes an acellular dermal matrix that comprises decellularized fish skin.

18. The wound treatment device according to claim 15, wherein the skin substitute comprises an extracellular matrix product in a three-dimensional form of particles, or a sheet, or a mesh.

19. The wound treatment device according to claim 15, wherein the contact film is a multi-layer film including a core layer, and first outer layer provided on a first surface of the core layer and a second outer layer provided on a second surface of the core layer, the first surface of the core layer being opposite from the second surface of the core layer, the first outer layer is in contact with and removable adhered to the skin substitute and the second outer layer is in contact with the absorbent core.

20. The wound treatment device according to claim 18, wherein the first outer layer is a silicone adhesive layer.
